SEQUENCE LISTING (89 sequences)

SEQ ID NO: 1 - protein, 9 aa, unidentified
  REGION 1..9: note = Murinae sp.
  REGION 1..9: note = misc_feature - Variable heavy chain CDR-H1 amino acid sequence of mouse antibody clones M14, M18, and M19
GDSITSGYW

SEQ ID NO: 2 - protein, 16 aa, unidentified
  REGION 1..16: note = Murinae sp.
  REGION 1..16: note = misc_feature - Variable heavy chain CDR-H2 amino acid sequence of mouse antibody clones M14 and M19
YISYSGITDYNPSLKS

SEQ ID NO: 3 - protein, 12 aa, unidentified
  REGION 1..12: note = Murinae sp.
  REGION 1..12: note = misc_feature - Variable heavy chain CDR-H3 amino acid sequence of mouse antibody clones M14, M18, and M19
ARYGNYGYAMDY

SEQ ID NO: 4 - protein, 25 aa, unidentified
  REGION 1..25: note = Murinae sp.
  REGION 1..25: note = misc_feature - Variable region heavy chain framework HC-FR1 amino acid sequence of mouse antibody clone M14
EVQLQESGPSLVKPSQTLSLTCSVT

SEQ ID NO: 5 - protein, 15 aa, unidentified
  REGION 1..15: note = Murinae sp.
  REGION 1..15: note = misc_feature - Variable region heavy chain framework HC-FR2 amino acid sequence of mouse antibody clone M14
NWIRKFPGNKLEYMG

SEQ ID NO: 6 - protein, 30 aa, unidentified
  REGION 1..30: note = Murinae sp.
  REGION 1..30: note = misc_feature - Variable region heavy chain framework HC-FR3 amino acid sequence of mouse antibody clone M14
RISITRDTSKNQYYLQLNSVTTEDTATYYC

SEQ ID NO: 7 - protein, 11 aa, unidentified
  REGION 1..11: note = Murinae sp.
  REGION 1..11: note = misc_feature - Variable region heavy chain framework HC-FR4 amino acid sequence of mouse antibody clone M14
WGQGTSVTVSS

SEQ ID NO: 8 - protein, 15 aa, unidentified
  REGION 1..15: note = Murinae sp.
  REGION 1..15: note = misc_feature - Variable light chain CDR-L1 amino acid sequence of mouse antibody clones M14 and M19
RASESVDTYGNSFMH

SEQ ID NO: 9 - protein, 7 aa, unidentified
  REGION 1..7: note = Murinae sp.
  REGION 1..7: note = misc_feature - Variable light chain CDR-L2 amino acid sequence of mouse antibody clones M14, M18, and M19
RASNLES

SEQ ID NO: 10 - protein, 9 aa, unidentified
  REGION 1..9: note = Murinae sp.
  REGION 1..9: note = misc_feature - Variable light chain CDR-L3 amino acid sequence of mouse antibody clones M14, M18, and M19
QQSYEDPWT

SEQ ID NO: 11 - protein, 23 aa, unidentified
  REGION 1..23: note = Murinae sp.
  REGION 1..23: note = misc_feature - Variable region light chain framework LC-FR1 amino acid sequence of mouse antibody clone M14
DIVLTQSPASLAVSLGQRATISC

SEQ ID NO: 12 - protein, 15 aa, unidentified
  REGION 1..15: note = Murinae sp.
  REGION 1..15: note = misc_feature - Variable region light chain framework LC-FR2 amino acid sequence of mouse antibody clone M14
WYQQKSGQSPKLLIY

SEQ ID NO: 13 - protein, 32 aa, unidentified
  REGION 1..32: note = Murinae sp.
  REGION 1..32: note = misc_feature - Variable region light chain framework LC-FR3 amino acid sequence of mouse antibody clone M14
GIPARFGGSGSRTDFTLTIDPVEADDVATYYC

SEQ ID NO: 14 - protein, 10 aa, unidentified
  REGION 1..10: note = Murinae sp.
  REGION 1..10: note = misc_feature - Variable region light chain framework LC-FR4 amino acid sequence of mouse antibody clone M14
FGGGTKLEIK

SEQ ID NO: 15 - protein, 118 aa, synthetic construct
  REGION 1..118: note = Synthetic: Caninized variable heavy chain amino acid sequence of mouse antibody clone M14
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSS

SEQ ID NO: 16 - protein, 111 aa, synthetic construct
  REGION 1..111: note = Synthetic: Caninized variable light chain amino acid sequence of mouse antibody clone M14
DIVMTQSPASLSVSLGQRATISCRASESVDTYGNSFMHWYQQKPGQSPKLLIYRASNLESGIPARFGGSGSGTDFTLTIDPVQADDVATYYCQQSYEDPWTFGGGTKLEIK

SEQ ID NO: 17 - protein, 448 aa, synthetic construct
  REGION 1..448: note = Synthetic: Caninized heavy chain sequence from mouse antibody clone M14 and canine IgG-A
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 18 - protein, 453 aa, synthetic construct
  REGION 1..453: note = Synthetic: Caninized heavy chain sequence from mouse antibody clone M14 and canine IgG-B
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 19 - protein, 451 aa, synthetic construct
  REGION 1..451: note = Synthetic: Caninized heavy chain sequence from mouse antibody clone M14 and canine IgG-C
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 20 - protein, 449 aa, synthetic construct
  REGION 1..449: note = Synthetic: Caninized heavy chain sequence from mouse antibody clone M14 and canine IgG-D
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 21 - protein, 221 aa, synthetic construct
  REGION 1..221: note = Synthetic: Caninized light chain sequence from mouse antibody clone M14 and canine light chain constant region
DIVMTQSPASLSVSLGQRATISCRASESVDTYGNSFMHWYQQKPGQSPKLLIYRASNLESGIPARFGGSGSGTDFTLTIDPVQADDVATYYCQQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 22 - protein, 159 aa, unidentified
  REGION 1..159: note = Canidae sp.
  REGION 1..159: note = misc_feature - Canine IL31 amino acid sequence
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 23 - protein, 17 aa, unidentified
  REGION 1..17: note = Canidae sp.
  REGION 1..17: note = misc_feature - Canine IL31 epitope
PSDVRKIILELQPLSRG

SEQ ID NO: 24 - protein, 111 aa, unidentified
  REGION 1..111: note = Murinae sp.
  REGION 1..111: note = misc_feature - Variable light chain amino acid sequence of mouse antibody clone M14
DIVLTQSPASLAVSLGQRATISCRASESVDTYGNSFMHWYQQKSGQSPKLLIYRASNLESGIPARFGGSGSRTDFTLTIDPVEADDVATYYCQQSYEDPWTFGGGTKLEIK

SEQ ID NO: 25 - protein, 118 aa, unidentified
  REGION 1..118: note = Murinae sp.
  REGION 1..118: note = misc_feature - Variable heavy chain amino acid sequence of mouse antibody clone M14
EVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSS

SEQ ID NO: 26 - protein, 221 aa, synthetic construct
  REGION 1..221: note = Synthetic: Chimeric variable light chain of mouse antibody clone M14 and canine light chain constant region
DIVLTQSPASLAVSLGQRATISCRASESVDTYGNSFMHWYQQKSGQSPKLLIYRASNLESGIPARFGGSGSRTDFTLTIDPVEADDVATYYCQQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 27 - protein, 453 aa, synthetic construct
  REGION 1..453: note = Synthetic: Chimeric variable heavy chain of mouse antibody clone M14 and canine IgG-B
EVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - protein, 159 aa, Felis catus
  REGION 1..159: note = misc_feature - Feline IL31 amino acid sequence NCBI ref: XP_011286140.1 [felis catus]
MLSHAGPARFALFLLCCMETLLPSHMAPAHRLQPSDVRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 29 - protein, 209 aa, Equus caballus
  REGION 1..209: note = misc_feature - Equine IL31 amino acid sequence
MVSHIGSTRFALFLLCCLGTLMFSHTGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLELAMKTLTTAEQALPPLDPSTPHAGAVALTHHQQDRTALDRAVFPFVWAAPRGGEVGDGGH

SEQ ID NO: 30 - protein, 221 aa, synthetic construct
  REGION 1..221: note = Synthetic: Chimeric variable light chain of mouse antibody clone M14 and feline light chain constant region
DIVLTQSPASLAVSLGQRATISCRASESVDTYGNSFMHWYQQKSGQSPKLLIYRASNLESGIPARFGGSGSRTDFTLTIDPVEADDVATYYCQQSYEDPWTFGGGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 31 - protein, 453 aa, synthetic construct
  REGION 1..453: note = Synthetic: Chimeric variable heavy chain of mouse antibody clone M14 and feline heavy chain constant region
EVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 32 - protein, 111 aa, synthetic construct
  REGION 1..111: note = Synthetic: Felinized variable light chain sequence from mouse antibody clone M14
EIQMTQSPSSLSASPGDRVTISCRASESVDTYGNSFMHWYQQKPGQSPKLLIYRASNLESGVPSRFSGSGSGTDFTLTISSLEPEDAATYYCQQSYEDPWTFGGGTKLEIK

SEQ ID NO: 33 - protein, 118 aa, synthetic construct
  REGION 1..118: note = Synthetic: Felinized variable heavy chain sequence from mouse antibody clone M14
DVQLVESGGDLVKPGGSLRLTCSVTGDSITSGYWNWVRQAPGKGLQWVAYISYSGITDYADSVKGRFTISRDNAKNTLYLQLNNLKAEDTATYYCARYGNYGYAMDYWGQGTLVTVSS

SEQ ID NO: 34 - protein, 221 aa, synthetic construct
  REGION 1..221: note = Synthetic: Felinized light chain sequence from mouse antibody clone M14
EIQMTQSPSSLSASPGDRVTISCRASESVDTYGNSFMHWYQQKPGQSPKLLIYRASNLESGVPSRFSGSGSGTDFTLTISSLEPEDAATYYCQQSYEDPWTFGGGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 35 - protein, 453 aa, synthetic construct
  REGION 1..453: note = Synthetic: Felinized heavy chain sequence from mouse antibody clone M14
DVQLVESGGDLVKPGGSLRLTCSVTGDSITSGYWNWVRQAPGKGLQWVAYISYSGITDYADSVKGRFTISRDNAKNTLYLQLNNLKAEDTATYYCARYGNYGYAMDYWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 36 - protein, 238 aa, unidentified
  REGION 1..238: note = Murinae sp.
  REGION 1..238: note = MISC_FEATURE - Light chain amino acid sequence of mouse antibody clone M14
METDTLLLWVLLLWVPGSTGDIVLTQSPASLAVSLGQRATISCRASESVDTYGNSFMHWYQQKSGQSPKLLIYRASNLESGIPARFGGSGSRTDFTLTIDPVEADDVATYYCQQSYEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - protein, 238 aa, unidentified
  REGION 1..238: note = Murinae sp.
  REGION 1..238: note = MISC_FEATURE - Light chain amino acid sequence of mouse antibody clone M18
METDTLLLWVLLLWVPGSTGDIVLTQSPASLAVSPGQRATISCRASESVDTYGNSFIHWYQQKPGQSPKLLIYRASNLESGIPARFSGSGSRTDFTLTINPVETDDVATYYCQQSYEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 238 aa, unidentified
  REGION 1..238: note = Murinae sp.
  REGION 1..238: note = MISC_FEATURE - Light chain amino acid sequence of mouse antibody clone M19
METDTLLLWVLLLWVPGSTGDIVLTQSPASLAVSLGQRATISCRASESVDTYGNSFMHWYQQKPGQPPKLLIYRASNLESGIPARFSGSGSRTDFTLTINPVEADDIATYYCQQSYEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 39 - protein, 238 aa, unidentified
  REGION 1..238: note = Murinae sp.
  REGION 1..238: note = MISC_FEATURE - Light chain amino acid sequence of mouse antibody clone M87
METDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 40 - protein, 152 aa, unidentified
  REGION 1..152: note = Murinae sp.
  REGION 1..152: note = MISC_FEATURE - Variable and hinge heavy chain amino acid sequence of mouse antibody clone M14
MAVLGLLLCLVTFPSCVLSEVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSSAKTTPPSVYPLAPGS

SEQ ID NO: 41 - protein, 152 aa, unidentified
  REGION 1..152: note = Murinae sp.
  REGION 1..152: note = MISC_FEATURE - Variable and hinge heavy chain amino acid sequence of mouse antibody clone M18
MAVLGLLFCLVTFPSCVLSEVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSSAKTTPPSVYPLAPGS

SEQ ID NO: 42 - protein, 152 aa, unidentified
  REGION 1..152: note = Murinae sp.
  REGION 1..152: note = MISC_FEATURE - Variable and hinge heavy chain amino acid sequence of mouse antibody clone M19
MAVLGLLFCLVTFPSCVLSEVQLQESGPSLVKPSQTLSLTCSVTGDSITSGYWNWIRKFPGNELEYMGYISYSGITYYNPSLKSRFSITRDTSKNQYYLQLNSVTTEDTATYYCARYGNYGYAMDYWGQGTSVTVSSAKTTPPSVYPLAPGS

SEQ ID NO: 43 - protein, 154 aa, unidentified
  REGION 1..154: note = Murinae sp.
  REGION 1..154: note = MISC_FEATURE - Variable and hinge heavy chain amino acid sequence of mouse antibody clone M87
MAVLGLLFCLVTFPSCVLSEVKLVESGGGLVQPGGSLRLSCATSGFTFTDYYMNWVRQPPGKALEWLGFIRNKANGYTTEYSASVKGRFTISRDNSQSILYLQMNTLRAEDSATYYCARDYYGSCFDYWGQGTTLTVSSAKTTPPSVYPLAPGS

SEQ ID NO: 44 - protein, 137 aa, unidentified
  REGION 1..137: note = Canidae sp.
  REGION 1..137: note = MISC_FEATURE - Mature canine IL31 amino acid sequence
SSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 45 - protein, 7 aa, synthetic construct
  REGION 1..7: note = IL31 epitope
  SITE 4: note = MISC_FEATURE - X is any amino acid; or wherein X is a hydrophobic amino acid, or wherein X is selected from A, V, I, and L, or wherein X is selected from V and I
  SITE 5: note = MISC_FEATURE - X is any amino acid; or wherein X is a hydrophilic amino acid, or wherein X is selected from A, R, K, Q, and N; or wherein X is selected from R and Q
PSDXXKI

SEQ ID NO: 46 - protein, 164 aa, Homo sapiens
  REGION 1..164: note = MISC_FEATURE - Human IL31 precursor amino acid sequence NCBI ref: NP_001014358.1
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 47 - protein, 177 aa, Odobenus rosmarus
  REGION 1..177: note = MISC_FEATURE - Predicted walrus IL31 amino acid sequence NCBI ref: XP_004395998.1 [odobeus rosmarus divergens]
  source 1..177: note = divergens
MLSHAGPARFALFLLCFMGTSLTSQTAPIHQLHPSDVRKIILELQPLSKGLLEDYLKKEMGVPESNHFLLPCLTSDSQPPRINSSAILPYFRAIRPLSDKNTINKIIEQLDKLKFQHEPETEVSVPADTFESKSFILAILQQFSACLDHVFKSLNPGPQQVMQGHLIEPIPSGTADV

SEQ ID NO: 48 - protein, 187 aa, Odobenus rosmarus
  REGION 1..187: note = MISC_FEATURE - Walrus_IL31_His6
  source 1..187: note = divergens
MLSHAGPARFALFLLCFMGTSLTSQTAPIHQLHPSDVRKIILELQPLSKGLLEDYLKKEMGVPESNHFLLPCLTSDSQPPRINSSAILPYFRAIRPLSDKNTINKIIEQLDKLKFQHEPETEVSVPADTFESKSFILAILQQFSACLDHVFKSLNPGPQQVMQGHLIEPIPSGTADVGSGSHHHHHH

SEQ ID NO: 49 - protein, 163 aa, Papio anubis
  REGION 1..163: note = MISC_FEATURE - Predicted olive baboon IL31 amino acid sequence NCBI ref: XP_003907358.1 [papio anubis]
MASHSGPATSVLFLLCCLGGWLTSHTLPVHFLQPSDIQKIVEELQSLSKMLLKDVKEDKGVLVSQNYTLPCLTPDAQPPNIIHSPAIRAYLKTIRELDNKSVIDEIIEHLDKLIFQDAPETNISVPTDTHECKRFILTISQQFSECMDLALKSLTSGAQQATT

SEQ ID NO: 50 - protein, 173 aa, Papio anubis
  REGION 1..173: note = MISC_FEATURE - Olive baboon_IL31_His6
MASHSGPATSVLFLLCCLGGWLTSHTLPVHFLQPSDIQKIVEELQSLSKMLLKDVKEDKGVLVSQNYTLPCLTPDAQPPNIIHSPAIRAYLKTIRELDNKSVIDEIIEHLDKLIFQDAPETNISVPTDTHECKRFILTISQQFSECMDLALKSLTSGAQQATTGSGSHHHHHH

SEQ ID NO: 51 - protein, 159 aa, Ursus maritimus
  REGION 1..159: note = MISC_FEATURE - Predicted polar bear IL31 amino acid sequence NCBI ref: XP_008687166.1 [ursus maritimus]
MLSRAVPAGFALFLLCYMETLLTSHTAPTHRLPPSDVRKIILELQPLSKGLLEDYLKKETGLPESNHSLVPCLTSDSEAPHINSSAILPYFRAIRPLSDKNVIDKIIEQLDKLKFQHEPETEVSVPADTFEGKSFILTILQQFSACLERVFKSLNPGAQ

SEQ ID NO: 52 - protein, 185 aa, Ursus maritimus
  REGION 1..185: note = MISC_FEATURE - Predicted weddell seal IL31 amino acid sequence NCBI ref: XP_006746595.1 [leptonychotes weddellii]
MLSHAGPARFALFLLCFMETSLTSQTVPIHQLQPSDVRKIILELQPLSKGLLEDYLKKEMGVPESNHFLLPCLTSDSQPPRINSSAILPYFRAIRPLSDKNIINKIIEQLDKLKFQHEPETEVSVPADTFESKSFILTILQQFSACLGHVLKSLNPGPQQVMQGHLAKPIPSGTADMYETLRHHH

SEQ ID NO: 53 - protein, 159 aa, Panthera tigris
  REGION 1..159: note = MISC_FEATURE - Predicted amur tiger IL31 amino acid sequence NCBI ref: XP_007079636.1 [panther tigris altaica]
  source 1..159: note = altaica
MLSHAGPARFALFLLCCMETLLPSHMAPTHRLQPSDVRKIILELRPMSKGLLQDYLKKEMGLPESNHSSLPCLSSDSQLPHINGSAILPYFRAIRPLSDKNTIDKIIEQLDKLKFQREPEAEVSMPADNFERKNFILAVLQQFSACLEHVLQSLNSGPQ

SEQ ID NO: 54 - protein, 159 aa, Acinonyx jubatus
  REGION 1..159: note = MISC_FEATURE - Predicted cheetah IL31 amino acid sequence NCBI ref: XP_014919275.1 [acinonyx jubatus]
MLSHAGPARFALFLLCCMETLLPSHMAPTHRLQPSDVRKIILELRPMSKGLLQDYLKKEMGLPESNHSSLPCLSSDSQLPHINGSAILPYFRAIRPLSDKNTIDKIIEQLDKLKFQREPEAEVSMPADNFERKNFILAVLQQFSACLEHVLQSLNSGSQ

SEQ ID NO: 55 - protein, 163 aa, Macaca fascicularis
  REGION 1..163: note = MISC_FEATURE - Predicted crab-eating macaque IL31 amino acid sequence GenBank ref: EHH66805.1 [macaca fascicularis]
MASHSGPATSVLFLLCCLGGWLTSHTLPVHFLQPSDIQKIVEELQSLSKMLLKDVKEDKGVLVSQNYTLPCLTPDAQPPNIIHSPAIRAYLKTIRQLDNKSVIDEIIEHLDKLIFQDAPETNISVPTDTHECKRFILTISQQFSECMDLALKSLTSGAQQATT

SEQ ID NO: 56 - protein, 158 aa, Macaca mulatta
  REGION 1..158: note = MISC_FEATURE - Predicted rhesus monkey IL31 amino acid sequence (partial) GenBank ref: EHH21279.1 [macaca mulatta]
GPATSVLFLLCCLGGWLTSHTLPVHFLQPSDIQKIVEELQSLSKMLLKDVKEDKGVLVSQNYTLPCLTPDAQPPNIIHSPAIRAYLKTIRQLDNKSVIDEIIEHLDKLIFQDAPETNISVPTDTHECKRFILTISQQFSECMDLALKSLTSGAQQATT

SEQ ID NO: 57 - protein, 163 aa, Mandrillus leucophaeus
  REGION 1..163: note = MISC_FEATURE - Predicted drill IL31 amino acid sequence NCBI ref: XP_011819882.1 [mandrillus leucophaeus]
MASHSGPATSVLFLLCCLGGWLTSHTLPVHFLQPSDIQKIVKELQSLSKMLLKDVKEDKGVLVSQNYTLPCLTPDAQPPNIIHSPAIRAYLKTIRQLDNRSVIDEIIEHLDKLIFQDAPETNISVPTDTHECKRFILTISQQFSECMDLALKSLTSGAQQATT

SEQ ID NO: 58 - protein, 163 aa, Chlorocebus sabaeus
  REGION 1..163: note = MISC_FEATURE - Predicted green monkey IL31 amino acid sequence NCBI ref: XP_008003211.1 [chlorocebus sabaeus]
MASHSGPATSVLFLLCCLGGWLTSHTLPVHFLQPSDIQKIVEELQSLSKMLLKDVKEDKGVLVSQNYKLPCLTPDAQPPNIIHSPAIRAYLKTIRQLDNKSVIDEIIEHLDKLIFQDAPETNISVPTDTHECKRFILTISQQFSECMDLALKSLTSGAQQATT

SEQ ID NO: 59 - protein, 163 aa, Cercocebus atys
  REGION 1..163: note = MISC_FEATURE - Predicted sooty mangebey IL31 amino acid sequence NCBI ref: XP_011926625.1 [cercocebus atys]
MASHSGPATSVLFLLCCLGGWLTSHTLPVHFLQPSDIQKIVEELQSLSKMLLKDVKEDKGVLVSQNYTLPCLTPDAQPPNIIHSPAIRAYLKTIRQLDNRSVIDEIIEHLDKLIFQDAPETNISVPTDTHECKRFILTISQQFSECMDLALKSLTSGAQQATT

SEQ ID NO: 60 - protein, 163 aa, Rhinopithecus roxellana
  REGION 1..163: note = MISC_FEATURE - Predicted golden snub-nosed monkey IL31 amino acid sequence NCBI ref: XP_010366647.1 [rhinopithecus roxellana]
MASHSGPTTSVLFLLCCLGGWLTSHTLPVHFLRPSDIQKIVEELQSLSKMLLKDVEEDKGVLVSQNYTLPCLTPDAQPPNIIHSPAIRAYLKTIRQLDNKSVIDEIIEHLDKLIFQDAPETNISVPTDTHECKRFILTISQQFSECMDLALKSLTSGAQQATT

SEQ ID NO: 61 - protein, 163 aa, Mus musculus
  REGION 1..163: note = MISC_FEATURE - Murine IL31 precursor amino acid sequence NCBI ref: NP_083870 [mus musculus]
MIFHTGTTKPTLVLLCCIGTWLATCSLSFGAPISKEDLRTTIDLLKQESQDLYNNYSIKQASGMSADESIQLPCFSLDREALTNISVIIAHLEKVKVLSENTVDTSWVIRWLTNISCFNPLNLNISVPGNTDESYDCKVFVLTVLKQFSNCMAELQAKDNTTC

SEQ ID NO: 62 - protein, 16 aa, unidentified
  REGION 1..16: note = Murinae sp.
  REGION 1..16: note = MISC_FEATURE - Variable heavy chain CDR-H2 amino acid sequence of mouse antibody clone M18
YISYSGITYYNPSLKS

SEQ ID NO: 63 - protein, 15 aa, unidentified
  REGION 1..15: note = Murinae sp.
  REGION 1..15: note = MISC_FEATURE - Variable light chain CDR-L1 amino acid sequence of mouse antibody clone M18
RASESVDTYGNSFIH

SEQ ID NO: 64 - protein, 131 aa, unidentified
  REGION 1..131: note = Murinae sp.
  REGION 1..131: note = MISC_FEATURE - Variable light chain amino acid sequence of mouse antibody clone M18
METDTLLLWVLLLWVPGSTGDIVLTQSPASLAVSPGQRATISCRASESVDTYGNSFIHWYQQKPGQSPKLLIYRASNLESGIPARFSGSGSRTDFTLTINPVETDDVATYYCQQSYEDPWTFGGGTKLEIK

SEQ ID NO: 65 - protein, 131 aa, unidentified
  REGION 1..131: note = Murinae sp.
  REGION 1..131: note = MISC_FEATURE - Variable light chain amino acid sequence of mouse antibody clone M19
METDTLLLWVLLLWVPGSTGDIVLTQSPASLAVSLGQRATISCRASESVDTYGNSFMHWYQQKPGQPPKLLIYRASNLESGIPARFSGSGSRTDFTLTINPVEADDIATYYCQQSYEDPWTFGGGTKLEIK

SEQ ID NO: 66 - protein, 131 aa, unidentified
  REGION 1..131: note = Murinae sp.
  REGION 1..131: note = MISC_FEATURE - Variable light chain amino acid sequence of mouse antibody clone M87
METDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSFGGGTKLEIK

SEQ ID NO: 67 - protein, 137 aa, unidentified
  REGION 1..137: note = Murinae sp.
  REGION 1..137: note = MISC_FEATURE - Variable heavy chain amino acid sequence of mouse antibody clone M18
MAVLGLLFCLVTFPSCVLSEVQLQESGPSLVKPSQTLSLTCSVTGDSITSGYWNWIRKFPGNKLEYMGYISYSGITDYNPSLKSRISITRDTSKNQYYLQLNSVTTEDTATYYCARYGNYGYAMDYWGQGTSVTVSS

SEQ ID NO: 68 - protein, 137 aa, unidentified
  REGION 1..137: note = Murinae sp.
  REGION 1..137: note = MISC_FEATURE - Variable heavy chain amino acid sequence of mouse antibody clone M19
MAVLGLLFCLVTFPSCVLSEVQLQESGPSLVKPSQTLSLTCSVTGDSITSGYWNWIRKFPGNELEYMGYISYSGITYYNPSLKSRFSITRDTSKNQYYLQLNSVTTEDTATYYCARYGNYGYAMDYWGQGTSVTVSS

SEQ ID NO: 69 - protein, 139 aa, unidentified
  REGION 1..139: note = Murinae sp.
  REGION 1..139: note = MISC_FEATURE - Variable heavy chain amino acid sequence of mouse antibody clone M87
MAVLGLLFCLVTFPSCVLSEVKLVESGGGLVQPGGSLRLSCATSGFTFTDYYMNWVRQPPGKALEWLGFIRNKANGYTTEYSASVKGRFTISRDNSQSILYLQMNTLRAEDSATYYCARDYYGSCFDYWGQGTTLTVSS

SEQ ID NO: 70 - protein, 25 aa, synthetic construct
  REGION 1..25: note = Caninized M14 HC-FR1
EVQLVESGPSLVKPGGSLRLTCSVT

SEQ ID NO: 71 - protein, 15 aa, synthetic construct
  REGION 1..15: note = Caninized M14 HC-FR2
NWIRKFPGNKLEYMG

SEQ ID NO: 72 - protein, 30 aa, synthetic construct
  REGION 1..30: note = Caninized M14 HC-FR3
RITISRDTSKNQYYLQLNSVTTEDTATYYC

SEQ ID NO: 73 - protein, 36 aa, synthetic construct
  REGION 1..36: note = Alternate caninized M14 HC-FR3
NPSLKSRITISRDTSKNQYYLQLNSVTTEDTATYYC

SEQ ID NO: 74 - protein, 11 aa, synthetic construct
  REGION 1..11: note = Caninized M14 HC-FR4
WGQGTLVTVSS

SEQ ID NO: 75 - protein, 23 aa, synthetic construct
  REGION 1..23: note = Caninized M14 LC-FR1
DIVMTQSPASLSVSLGQRATISC

SEQ ID NO: 76 - protein, 15 aa, synthetic construct
  REGION 1..15: note = Caninized M14 LC-FR2
WYQQKPGQSPKLLIY

SEQ ID NO: 77 - protein, 32 aa, synthetic construct
  REGION 1..32: note = Caninized M14 LC-FR3
GIPARFGGSGSGTDFTLTIDPVQADDVATYYC

SEQ ID NO: 78 - protein, 10 aa, synthetic construct
  REGION 1..10: note = Caninized M14 LC-FR4
FGGGTKLEIK

SEQ ID NO: 79 - protein, 25 aa, synthetic construct
  REGION 1..25: note = Felinized M14 HC-FR1
DVQLVESGGDLVKPGGSLRLTCSVT

SEQ ID NO: 80 - protein, 15 aa, synthetic construct
  REGION 1..15: note = Felinized M14 HC-FR2
NWVRQAPGKGLQWVA

SEQ ID NO: 81 - protein, 37 aa, synthetic construct
  REGION 1..37: note = Felinized M14 HC-FR3
ADSVKGRFTISRDNAKNTLYLQLNNLKAEDTATYYCA

SEQ ID NO: 82 - protein, 11 aa, synthetic construct
  REGION 1..11: note = Felinized M14 HC-FR4
WGQGTLVTVSS

SEQ ID NO: 83 - protein, 23 aa, synthetic construct
  REGION 1..23: note = Felinized M14 LC-FR1
EIQMTQSPSSLSASPGDRVTISC

SEQ ID NO: 84 - protein, 15 aa, synthetic construct
  REGION 1..15: note = Felinized M14 LC-FR2
WYQQKPGQSPKLLIY

SEQ ID NO: 85 - protein, 32 aa, synthetic construct
  REGION 1..32: note = Felinized M14 LC-FR3
GVPSRFSGSGSGTDFTLTISSLEPEDAATYYC

SEQ ID NO: 86 - protein, 10 aa, synthetic construct
  REGION 1..10: note = Felinized M14 LC-FR4
FGGGTKLEIK

SEQ ID NO: 87 - protein, 10 aa, unidentified
  REGION 1..10: note = Murinae sp.
  REGION 1..10: note = MISC_FEATURE - Alternate variable heavy chain CDR-H2 amino acid sequence of mouse antibody clone M18
YISYSGITYY

SEQ ID NO: 88 - protein, 7 aa, unidentified
  REGION 1..7: note = Canidae sp.
  REGION 1..7: note = MISC_FEATURE - Alternative canine IL31 epitope
PSDVRKI

SEQ ID NO: 89 - protein, 10 aa, unidentified
  REGION 1..10: note = Murinae sp.
  REGION 1..10: note = MISC_FEATURE - Alternate variable heavy chain CDR-H2 amino acid sequence of mouse antibody clones M14 and M19
YISYSGITDY

(19) United States
(12) Patent Application Publication
(10) Pub. No.: US 2024/0101660 A1
(43) Pub. Date: Mar. 28, 2024

(54) ANTI-IL31 ANTIBODIES FOR VETERINARY USE

(71) Applicant: Elanco US Inc., Greenfield, IN (US)

(72) Inventors: Shyr Jiann Li, Burlingame, CA (US); Lam Nguyen, Burlingame, CA (US); Hangjun Zhan, Burlingame, CA (US)

(21) Appl. No.: 18/311,777

(22) Filed: May 3, 2023

Related U.S. Application Data
(63) Continuation of application No. 16/488,045, filed on Aug. 22, 2019, now Pat. No. 11,673,946.
(60) Provisional application No. 62/463,543, filed on Feb. 24, 2017.

(51) Int. Cl. C07K 16/24

(52) U.S. Cl. CPC C07K 16/244; A61K 2039/552; C07K 2317/24; C07K 2317/33; C07K 2317/34; C07K 2317/52; C07K 2317/56; C07K 2317/565; C07K 2317/76; C07K 2317/92

(57) ABSTRACT

Provided are various embodiments relating to anti-IL31 antibodies binding to canine IL31. Such antibodies can be used in methods to treat IL31-induced conditions in companion animals, such as canines, felines, and equines.

### CROSS-REFERENCE TO RELATED APPLICATIONS

This application is a Divisional Application of U.S. application Ser. No. 16/488,045, filed 22 Aug. 2019, which is a National Stage Application of PCT/US2018/017623, filed 9 Feb. 2018, which claims the benefit of U.S. Provisional Application No. 62/463,543, filed 24 Feb. 2017, and claims the benefit of International Application No. PCT/US2017/023788, filed 23 Mar. 2017, the entire contents of each of which are incorporated herein by reference.

### REFERENCE TO SEQUENCE LISTING SUBMITTED AS A COMPLIANT ASCII TEXT FILE (.xml)

A Sequence Listing is submitted herewith as an ASCII compliant text file named “2920951-451006_Sequence_Listing_ST26” created on 11 Dec. 2023 and having a size of 142,666 bytes as permitted under 37 C.F.R. § 1.821 (c). The material in the aforementioned file is hereby incorporated by reference in its entirety.

### BACKGROUND OF THE INVENTION

### Field

This invention relates to isolated anti-IL31 antibodies, for example, binding to canine IL31, and methods of using the same, for example, treating IL31-induced conditions or reducing IL31 signaling function in cells, for instance in companion animals, such as canines, felines, and equines.

Interleukin 31 (IL31) is a cytokine mostly produced by Th2 cells and understood to be involved in promoting skin disease, such as pruritic and other forms of allergic diseases (for example, atopic dermatitis). IL31 functions by binding its receptor and activating downstream activities, such as activation of JAK1, and is thought to cause many of the clinical problems associated with dermatitis and other disorders.

Companion animals such as cats, dogs, and horses, suffer from many skin diseases similar to human skin diseases, including atopic dermatitis. However, the IL31 sequence is divergent between human, cat, dog, and horse. There remains a need, therefore, for methods and compounds that can be used specifically to bind companion animal IL31 for treating IL31-induced conditions and for reducing IL31 signaling.

### SUMMARY OF THE INVENTION

In some embodiments, an isolated antibody is provided that binds to canine IL31. In some embodiments the antibody binds to an epitope comprising amino acids 34-50 of SEQ ID NO: 22. In some embodiments, the antibody binds to an epitope comprising the amino acid sequence of SEQ ID NO: 23. In some embodiments the antibody binds to an epitope comprising the amino acid sequence of PSDX₁×₂KI (SEQ ID NO: 45), wherein X is any amino acid residue. In some embodiments, X₁ is a hydrophobic amino acid. In some embodiments, X₁ is selected from A, V, I, and L. In some embodiments, X₁ is selected from V and I. In some embodiments, X₂ is a hydrophilic amino acid. In some embodiments, X₂ is selected from A, R, K, Q, and N. In some embodiments, X₂ is selected from R and Q. In some embodiments, X₁ is V and X₂ is R. In some embodiments, X₁ is I and X₂ is Q. In some embodiments, the antibody binds to an epitope comprising the amino acid sequence of SEQ ID NO: 88.

In some embodiments, the antibody binds to canine IL31 with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, the antibody reduces IL31 signaling function in a companion animal species, as measured by a reduction in STAT-3 phosphorylation. In some embodiments, the companion animal species is canine, feline, or equine.

In some embodiments, the antibody binds to feline IL31 or equine IL31, as determined by immunoblot analysis and/or biolayer interferometry. In some embodiments, the antibody competes with monoclonal M14 antibody in binding to canine IL31. In some embodiments, the antibody competes with monoclonal M14 antibody in binding to feline IL31. In some embodiments, the antibody does not bind to human IL31 as determined by immunoblot analysis and/or biolayer interferometry.

In some embodiments, the antibody is a monoclonal antibody. In some embodiments, the antibody is a canine, a caninized, a feline, a felinized, an equine, an equitized, or a chimeric antibody. In some embodiments, the antibody is a chimeric antibody comprising murine variable heavy chain framework regions or murine variable light chain framework regions.

In some embodiments, the antibody comprises a heavy chain and a light chain, wherein:
a. the heavy chain comprises a CDR-H1 sequence having at least 85% sequence identity, at least 90% sequence identity, at least 95% sequence identity, or at least 98% sequence identity to the amino acid sequence of SEQ ID NO: 1; a CDR-H2 sequence having at least 85% sequence identity, at least 90% sequence identity, at least 95% sequence identity, or at least 98% sequence identity to the amino acid sequence of SEQ ID NO: 2, 62, 89, or 87; and a CDR-H3 sequence having at least 85% sequence identity, at least 90% sequence identity, at least 95% sequence identity, or at least 98% sequence identity to the amino acid sequence of SEQ ID NO: 3, and b. the light chain comprises a CDR-L1 sequence having at least 85% sequence identity, at least 90% sequence identity, at least 95% sequence identity, or at least 98% sequence identity to the amino acid sequence of SEQ ID NO: 8 or SEQ ID NO: 63; a CDR-L2 sequence having at least 85% sequence identity, at least 90% sequence identity, at least 95% sequence identity, or at least 98% sequence identity to the amino acid sequence of SEQ ID NO: 9; and a CDR-L3 sequence having at least 85% sequence identity, at least 90% sequence identity, at least 95% sequence identity, or at least 98% sequence identity to the amino acid sequence of SEQ ID NO: 10.

In some embodiments, the antibody comprises a heavy chain comprising (a) a CDR-H1 comprising the amino acid sequence of SEQ ID NO: 1, (b) a CDR-H2 comprising the amino acid sequence of SEQ ID NO: 2 or 89, and (c) a CDR-H3 comprising the amino acid sequence of SEQ ID NO: 3.

In some embodiments, the antibody comprises a heavy chain comprising (a) a CDR-H1 comprising the amino acid sequence of SEQ ID NO: 1, (b) a CDR-H2 comprising the amino acid sequence of SEQ ID NO: 62 or 87, and (c) a CDR-H3 comprising the amino acid sequence of SEQ ID NO: 3.

In some embodiments, the antibody comprises a light chain comprising (a) a CDR-L1 comprising the amino acid sequence of SEQ ID NO: 8, (b) a CDR-L2 comprising the amino acid sequence of SEQ ID NO: 9, and (c) a CDR-L3 comprising the amino acid sequence of SEQ ID NO: 10.

In some embodiments, the antibody comprises a light chain comprising (a) a CDR-L1 comprising the amino acid sequence of SEQ ID NO: 63, (b) a CDR-L2 comprising the amino acid sequence of SEQ ID NO: 9, and (c) a CDR-L3 comprising the amino acid sequence of SEQ ID NO: 10.

In some embodiments, the antibody comprises one or more of (a) a variable region heavy chain framework 1 (HC-FR1) sequence of SEQ ID NO: 4, 70, or 79, (b) a HC-FR2 sequence of SEQ ID NO: 5, 71, or 80, (c) a HC-FR3 sequence of SEQ ID NO: 6, 72, 73, or 81, (d) a HC-FR4 sequence of SEQ ID NO: 7, 74, or 82, (e) a variable region light chain framework 1 (LC-FR1) sequence of SEQ ID NO: 11, 75, or 83, (f) an LC-FR2 sequence of SEQ ID NO: 12, 76, or 84, (g) an LC-FR3 sequence of SEQ ID NO: 13, 77, or 85, or (h) an LC-FR4 sequence of SEQ ID NO: 14, 78, or 86.

In some embodiments, the antibody comprises:
a. (i) a variable light chain sequence having at least 85%, at least 90%, at least 95%, or at least 98% sequence identity to the amino acid sequence of SEQ ID NO: 24; (ii) a variable heavy chain sequence having at least 85%, at least 90%, at least 95%, or at least 98% sequence identity to the amino acid sequence of SEQ ID NO: 25; or (iii) a variable light chain sequence as in (i) and a variable heavy chain sequence as in (ii); or
b. (i) a variable light chain sequence having at least 85%, at least 90%, at least 95%, or at least 98% sequence identity to the amino acid sequence of SEQ ID NO: 16; (ii) a variable heavy chain sequence having at least 85%, at least 90%, at least 95%, or at least 98% sequence identity to the amino acid sequence of SEQ ID NO: 15; or (iii) a variable light chain sequence as in (i) and a variable heavy chain sequence as in (ii); or
c. (i) a variable light chain sequence having at least 85%, at least 90%, at least 95%, or at least 98% sequence identity to the amino acid sequence of SEQ ID NO: 32; (ii) a variable heavy chain sequence having at least 85%, at least 90%, at least 95%, or at least 98% sequence identity to the amino acid sequence of SEQ ID NO: 33; or (iii) a variable light chain sequence as in (i) and a variable heavy chain sequence as in (ii).

In some embodiments, the antibody comprises a variable light chain sequence of SEQ ID NO: 24; SEQ ID NO: 16; or SEQ ID NO: 32. In some embodiments, the antibody comprises a variable heavy chain sequence SEQ ID NO: 25; SEQ ID NO: 15; or SEQ ID NO: 33. In some embodiments, the antibody comprises: a variable light chain sequence of SEQ ID NO: 24 and a variable heavy chain sequence of SEQ ID NO: 25; a variable light chain sequence of SEQ ID NO: 16 and a variable heavy chain sequence of SEQ ID NO: 15; or a variable light chain sequence of SEQ ID NO: 32 and a variable heavy chain sequence of SEQ ID NO: 33.

In some embodiments, the antibody is a chimeric antibody comprising a constant heavy chain region or constant light chain region derived from a companion animal.

In some embodiments, the antibody comprises (a) a canine heavy chain constant region selected from an IgG-A, IgG-B, IgG-C, and IgG-D constant region; (b) a feline heavy chain constant region selected from an IgG1, IgG2a, and IgG2b constant region; or (c) an equine heavy chain constant region selected from an IgG1, IgG2, IgG3, IgG4, IgG5, IgG6 and IgGγ constant region.

In some embodiments, the antibody comprises:
a. (i) a light chain amino acid sequence of SEQ ID NO: 26; (ii) a heavy chain amino acid sequence of SEQ ID NO: 27; or (iii) a light chain amino acid sequence as in (i) and a heavy chain amino acid sequence as in (ii); or
b. (i) a light chain amino acid sequence of SEQ ID NO: 30; (ii) a heavy chain amino acid sequence of SEQ ID NO: 31; or (iii) a light chain amino acid sequence as in (i) and a heavy chain amino acid sequence as in (ii); or
c. (i) a light chain amino acid sequence of SEQ ID NO: 34; (ii) a heavy chain amino acid sequence of SEQ ID NO: 35, or (iii) a light chain amino acid sequence as in (i) and a heavy chain amino acid sequence as in (ii).

In some embodiments, the antibody comprises a light chain amino acid sequence of SEQ ID NO: 21. In some embodiments, the antibody comprises a heavy chain amino acid sequence of SEQ ID NO: 17; SEQ ID NO: 18; SEQ ID NO: 19; or SEQ ID NO: 20.

In some embodiments, the antibody is an antibody fragment selected from Fv, scFv, Fab, Fab′, F(ab′)2, and Fab′-SH.

In some embodiments, the antibody is bi-specific, wherein the antibody binds to IL31 and one or more antigens selected from IL17, TNFα, CD20, CD19, CD25, IL4, IL13, IL23, IgE, CD11α, IL6R, α4-Intergrin, IL12, IL1β, or BlyS.

In some embodiments, an isolated nucleic acid is provided, which encodes an anti-IL31 antibody described herein above. In some embodiments, a host cell is provided, which comprises a nucleic acid encoding an anti-IL31 antibody described herein above. In some embodiments, a method of producing an anti-IL31 antibody is provided, which comprises culturing such a host cell comprising a nucleic acid encoding an anti-IL31 antibody described herein above and isolating the antibody.

In some embodiments, a pharmaceutical composition is provided, which comprises an anti-IL31 antibody described herein and a pharmaceutically acceptable carrier. In some embodiments, a pharmaceutical composition is provided, which comprises an anti-IL31 antibody described herein and a pharmaceutically acceptable carrier, wherein the pharmaceutically acceptable carrier comprises L-histidine, sodium chloride, and polysorbate 80.

In some embodiments, the pharmaceutical composition has a pH of from 5.0 to 6.2. In some embodiments, the pharmaceutical composition has a pH of from 5.0 to 6.0, or from 5.3 to 5.7, or 5.5.

In some embodiments, the pharmaceutical composition has an L-histidine concentration of from 5 mM to 100 mM, from 10 mM to 50 mM, from 20 mM to 30 mM, from 10 to 30 mM, or 20 mM.

In some embodiments, the pharmaceutical composition has a sodium chloride concentration of from 80 to 200 mM, from 100 to 175 mM, from 120 to 150 mM, or 140 mM.

In some embodiments, the pharmaceutical composition has a polysorbate 80 concentration of from 0.005 mg/mL to 0.5 mg/mL, from 0.01 mg/mL to 0.1 mg/mL, or 0.05 mg/mL.

In some embodiments, the pharmaceutically acceptable carrier comprises at least one sugar. In some embodiments, the pharmaceutical composition has a concentration of at least one sugar of from 0.5% to 20%, from 1% to 10%, from 1% to 5%, or from 1% to 3%. In some embodiments, the pharmaceutically acceptable carrier comprises sucrose, trehalose, D-mannitol, maltose, and/or sorbitol.

In some embodiments, the pharmaceutically acceptable carrier comprises an anti-bacterial agent. In some embodiments, the pharmaceutical composition comprises m-cresol or methylparaben. In some embodiments, the pharmaceutical composition comprises 0.2% m-cresol and/or 0.9% methylparaben.

Uses of Antibodies and Pharmaceutical Compositions

In some embodiments, methods of treating a companion animal species having an IL31-induced condition are provided, comprising administering to the companion animal species a therapeutically effective amount of an anti-1131 antibody described herein or a pharmaceutical composition comprising the antibody described herein. In some embodiments, the companion animal species is canine, feline, or equine. In some embodiments, the IL31-induced condition is a pruritic or allergic condition. In some embodiments, the IL31-induced condition is selected from atopic dermatitis, pruritus, asthma, psoriasis, scleroderma and eczema.

In some embodiments, the anti-IL31 antibody or the pharmaceutical composition is administered parenterally. In some embodiments, the anti-IL31 antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

In some embodiments, the method comprises administering in combination with the anti-IL31 antibody or the pharmaceutical composition a Jak inhibitor, a PI3K inhibitor, an AKT inhibitor, or a MAPK inhibitor. In some embodiments, the method comprises administering in combination with the anti-IL31 antibody or the pharmaceutical composition one or more antibodies selected from an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11α antibody, anti-IL6R antibody, anti-α4-Intergrin antibody, an anti-IL12 antibody, an anti-IL1β antibody, and an anti-BlyS antibody.

In some embodiments, methods of reducing IL31 signaling function in a cell are provided, comprising exposing to the cell an anti-IL31 antibody the pharmaceutical composition described herein under conditions permissive for binding of the antibody to extracellular IL31, thereby reducing binding to IL31 receptor and/or reducing IL31 signaling function by the cell. In some embodiments, the cell is exposed to the antibody or the pharmaceutical composition ex vivo. In some embodiments, the cell is exposed to the antibody or the pharmaceutical composition in vivo. In some embodiments, the cell is a canine cell, a feline cell, or an equine cell.

In some embodiments, a method for detecting IL31 in a sample from a companion animal species are provided, comprising contacting the sample with an anti-IL31 antibody or the pharmaceutical composition described herein under conditions permissive for binding of the antibody to IL31, and detecting whether a complex is formed between the antibody and IL31 in the sample. In some embodiments, the sample is a biological sample obtained from a canine, a feline, or an equine.

### BRIEF DESCRIPTION OF THE DRAWINGS

FIG. 1A is an alignment of variable light sequences of M14, M18, M19, and M87 mouse monoclonal antibody clones. FIG. 1 the term “atnB is an alignment of variable heavy sequences of M14, M18, M19, and M87 mouse monoclonal antibody clones.

FIG. 2A and FIG. 2B are graphs of canine IL31 binding analysis with varying concentrations of chimeric M14 antibody.

FIG. 3A and FIG. 3B are graphs of canine IL31 binding analysis with varying concentrations of caninized M14 antibody.

FIG. 4 is an immunoblot showing inhibited canine IL31 signaling at varying concentrations of caninized M14 antibody.

FIGS. 5A and 5B are immunoblots of GST-canine-IL31 deletions probed with M14 antibody and anti-GST antibody, respectively.

FIGS. 6A and 6B are immunoblots of GST-canine-IL31 deletions probed with M14 antibody and anti-GST antibody, respectively.

FIGS. 7A and 7B are immunoblots of feline and equine IL31 proteins fused to human Fc probed with M14 antibody and anti-FC antibody, respectively.

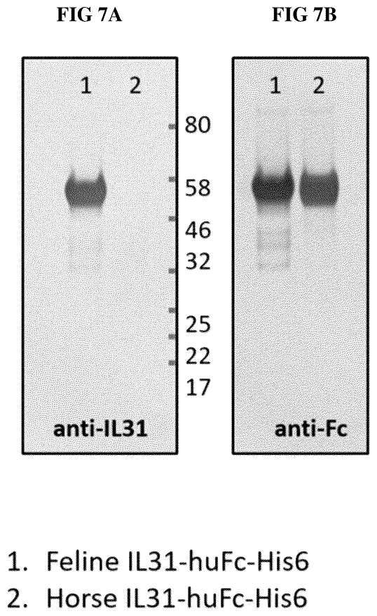

FIG. 8 shows immunoblot analysis of fine epitope mapping and alanine scanning of mature canine IL31 epitope using anti-canine IL31 antibody (top panel) and anti-GST antibody (bottom panel).

FIG. 9 shows immunoblot analysis of fine epitope mapping and alanine scanning of mature canine IL31 epitope using anti-canine IL31 antibody (top panel) and anti-GST antibody (bottom panel).

FIG. 10 shows immunoblot analysis of fine epitope mapping and alanine scanning of mature canine IL31 epitope using anti-canine IL31 antibody (top panel) and anti-GST antibody (bottom panel).

FIG. 11 shows immunoblot analysis of fine epitope mapping and alanine scanning of mature canine IL31 epitope using anti-canine IL31 antibody (top panel) and anti-GST antibody (bottom panel).

FIG. 12 shows immunoblot analysis of fine epitope mapping and alanine scanning of mature canine IL31 epitope using anti-canine IL31 antibody (top panel) and anti-GST antibody (bottom panel).

FIG. 13 is an immunoblot cross reactivity of anti-canine IL31 antibody M14 to walrus IL31.

### DESCRIPTION OF CERTAIN SEQUENCES

Table 1 provides a listing of certain sequences referenced herein.

TABLE 1
Description of Certain Sequences

| SEQ ID | | |
|---|---|---|
| NO: | SEQUENCE | DESCRIPTION |
| 1 | GDSITSGYW | Variable heavy chain CDR-H1 |
| | | amino acid sequence of mouse |
| | | antibody clones M14, M18, |
| | | and M19 |
| 2 | YISYSGITDYNPSLKS | Variable heavy chain CDR-H2 |
| | | amino acid sequence of mouse |
| | | antibody clones M14 and M19 |
| 62 | YISYSGITYYNPSLKS | Variable heavy chain CDR-H2 |
| | | amino acid sequence of mouse |
| | | antibody clone M18 |
| 89 | YISYSGITDY | Alternate variable heavy chain |
| | | CDR-H2 amino acid sequence |
| | | of mouse antibody clones M14 |
| | | and M19 |
| 87 | YISYSGITYY | Alternate variable heavy chain |
| | | CDR-H2 amino acid sequence |
| | | of mouse antibody clone M18 |
| 3 | ARYGNYGYAMDY | Variable heavy chain CDR-H3 |
| | | amino acid sequence of mouse |
| | | antibody clones M14, M18, |
| | | and M19 |
| 4 | EVQLQESGPSLVKPSQTLSLTCSVT | Variable region heavy chain |
| | | framework HC-FR1 amino |
| | | acid sequence of mouse |
| | | antibody clone M14 |
| 5 | NWIRKFPGNKLEYMG | Variable region heavy chain |
| | | framework HC-FR2 amino |
| | | acid sequence of mouse |
| | | antibody clone M14 |
| 6 | RISITRDTSKNQYYLQLNSVTTEDTATYYC | Variable region heavy chain |
| | | framework HC-FR3 amino |
| | | acid sequence of mouse |
| | | antibody clone M14 |
| 7 | WGQGTSVTVSS | Variable region heavy chain |
| | | framework HC-FR4 amino |
| | | acid sequence of mouse |
| | | antibody clone M14 |
| 8 | RASESVDTYGNSFMH | Variable light chain CDR-L1 |
| | | amino acid sequence of mouse |
| | | antibody clones M14 and M19 |
| 63 | RASESVDTYGNSFIH | Variable light chain CDR-L1 |
| | | amino acid sequence of mouse |
| | | antibody clone M18 |
| 9 | RASNLES | Variable light chain CDR-L2 |
| | | amino acid sequence of mouse |
| | | antibody clones M14, M18, |
| | | and M19 |
| 10 | QQSYEDPWT | Variable light chain CDR-L3 |
| | | amino acid sequence of mouse |
| | | antibody clones M14, M18, |
| | | and M19 |
| 11 | DIVLTQSPASLAVSLGQRATISC | Variable region light chain |
| | | framework LC-FR1 amino |
| | | acid sequence of mouse |
| | | antibody clone M14 |
| 12 | WYQQKSGQSPKLLIY | Variable region light chain |
| | | framework LC-FR2 amino |
| | | acid sequence of mouse |
| | | antibody clone M14 |
| 13 | GIPARFGGSGSRTDFTLTIDPVEADDVATYYC | Variable region light chain |
| | | framework LC-FR3 amino |
| | | acid sequence of mouse |
| | | antibody clone M14 |
| 14 | FGGGTKLEIK | Variable region light chain |
| | | framework LC-FR4 amino |
| | | acid sequence of mouse |
| | | antibody clone M14 |
| 15 | EVQLVESGPSLVKPGGSLRLTCSVTGDSITSGYWNWI | Caninized variable heavy chain |
| | RKFPGNKLEYMGYISYSGITDYNPSLKSRITISRDTS | amino acid sequence of mouse |
| | KNQYYLQLNSVTTEDTATYYCARYGNYGYAMDYWGQG | antibody clone M14 |
| | TLVTVSS | |
| 16 | DIVMTQSPASLSVSLGQRATISCRASESVDTYGNSFM | Caninized variable light chain |
| | HWYQQKPGQSPKLLIYRASNLESGIPARFGGSGSGTD | amino acid sequence of mouse |
| | FTLTIDPVQADDVATYYCQQSYEDPWTFGGGTKLEIK | antibody clone M14 |
| 70 | EVQLVESGPSLVKPGGSLRLTCSVT | Caninized M14 HC-FR1 |
| 71 | NWIRKFPGNKLEYMG | Caninized M14 HC-FR2 |
| 72 | RITISRDTSKNQYYLQLNSVTTEDTATYYC | Caninized M14 HC-FR3 |
| 73 | NPSLKSRITISRDTSKNQYYLQLNSVITEDTATYYC | Alternate caninized M14 HC- |
| | | FR3 |
| 74 | WGQGTLVTVSS | Caninized M14 HC-FR4 |
| 75 | DIVMTQSPASLSVSLGQRATISC | Caninized M14 LC-FR1 |
| 76 | WYQQKPGQSPKLLIY | Caninized M14 LC-FR2 |
| 77 | GIPARFGGSGSGTDFTLTIDPVQADDVATYYC | Caninized M14 LC-FR3 |
| 78 | FGGGTKLEIK | Caninized M14 LC-FR4 |
| 17 | EVQLVESGPSLVKPGGSLRLTCSVTGDSITSGYWNWI | Caninized heavy chain |
| | RKFPGNKLEYMGYISYSGITDYNPSLKSRITISRDTS | sequence from mouse antibody |
| | KNQYYLQLNSVTTEDTATYYCARYGNYGYAMDYWGQG | clone M14 and canine IgG-A |
| | TLVTVSSASTTAPSVFPLAPSCGSTSGSTVALACLVS | |
| | GYFPEPVTVSWNSGSLTSGVHTFPSVLQSSGLHSLSS | |
| | MVTVPSSRWPSETFTCNVVHPASNTKVDKPVFNECRC | |
| | TDTPCPVPEPLGGPSVLIFPPKPKDILRITRIPEVIC | |
| | VVLDLGREDPEVQISWFVDGKEVHTAKTQSREQQFNG | |
| | TYRVVSVLPIEHQDWLTGKEFKCRVNHIDLPSPIERT | |
| | ISKARGRAHKPSVYVLPPSPKELSSSDTVSITCLIKD | |
| | FYPPDIDVEWQSNGQQEPERKHRMTPPQLDEDGSYFL | |
| | YSKLSVDKSRWQQGDPFTCAVMHETLQNHYTDLSLSH | |
| | SPGK | |
| 18 | EVQLVESGPSLVKPGGSLRLTCSVTGDSITSGYWNWI | Caninized heavy chain |
| | RKFPGNKLEYMGYISYSGITDYNPSLKSRITISRDTS | sequence from mouse antibody |
| | KNQYYLQLNSVTTEDTATYYCARYGNYGYAMDYWGQG | clone M14 and canine IgG-B |
| | TLVTVSSASTTAPSVFPLAPSCGSTSGSTVALACLVS | |
| | GYFPEPVTVSWNSGSLTSGVHTFPSVLQSSGLYSLSS | |
| | MVTVPSSRWPSETFTCNVAHPASKTKVDKPVPKRENG | |
| | RVPRPPDCPKCPAPEMLGGPSVFIFPPKPKDTLLIAR | |
| | TPEVTCVVVDLDPEDPEVQISWFVDGKQMQTAKTQPR | |
| | EEQFNGTYRVVSVLPIGHQDWLKGKQFTCKVNNKALP | |
| | SPIERTISKARGQAHQPSVYVLPPSREELSKNTVSLT | |
| | CLIKDFFPPDIDVEWQSNGQQEPESKYRTTPPQLDED | |
| | GSYFLYSKLSVDKSRWQRGDTFICAVMHEALHNHYTQ | |
| | ESLSHSPGK | |
| 19 | EVQLVESGPSLVKPGGSLRLTCSVTGDSITSGYWNWI | Caninized heavy chain |
| | RKFPGNKLEYMGYISYSGITDYNPSLKSRITISRDTS | sequence from mouse antibody |
| | KNQYYLQLNSVTTEDTATYYCARYGNYGYAMDYWGQG | clone M14 and canine IgG-C |
| | TLVTVSSASTTAPSVFPLAPSCGSQSGSTVALACLVS | |
| | GYIPEPVTVSWNSVSLTSGVHTFPSVLQSSGLYSLSS | |
| | MVTVPSSRWPSETFTCNVAHPATNTKVDKPVAKECEC | |
| | KCNCNNCPCPGCGLLGGPSVFIFPPKPKDILVTARTP | |
| | TVTCVVVDLDPENPEVQISWFVDSKQVQTANTQPREE | |
| | QSNGTYRVVSVLPIGHQDWLSGKQFKCKVNNKALPSP | |
| | IEEIISKTPGQAHQPNVYVLPPSRDEMSKNIVTLICL | |
| | VKDFFPPEIDVEWQSNGQQEPESKYRMTPPQLDEDGS | |
| | YFLYSKLSVDKSRWQRGDTFICAVMHEALHNHYTQIS | |
| | LSHSPGK | |
| 20 | EVQLVESGPSLVKPGGSLRLTCSVTGDSITSGYWNWI | Caninized heavy chain |
| | RKFPGNKLEYMGYISYSGITDYNPSLKSRITISRDTS | sequence from mouse antibody |
| | KNQYYLQLNSVTTEDTATYYCARYGNYGYAMDYWGQG | clone M14 and canine IgG-D |
| | TLVTVSSASTTAPSVFPLAPSCGSTSGSTVALACLVS | |
| | GYFPEPVTVSWNSGSLTSGVHTFPSVLQSSGLYSLSS | |
| | TVTVPSSRWPSETFTCNVVHPASNTKVDKPVPKESTC | |
| | KCISPCPVPESLGGPSVFIFPPKPKDILRITRIPEIT | |
| | CVVLDLGREDPEVQISWFVDGKEVHTAKTQPREQQFN | |
| | STYRVVSVLPIEHQDWLTGKEFKCRVNHIGLPSPIER | |
| | TISKARGQAHQPSVYVLPPSPKELSSSDTVTLICLIK | |
| | DFFPPEIDVEWQSNGQPEPESKYHTTAPQLDEDGSYF | |
| | LYSKLSVDKSRWQQGDTFTCAVMHEALQNHYTDLSLS | |
| | HSPGK | |
| 21 | DIVMTQSPASLSVSLGQRATISCRASESVDTYGNSFM | Caninized light chain sequence |
| | HWYQQKPGQSPKLLIYRASNLESGIPARFGGSGSGTD | from mouse antibody clone |
| | FTLTIDPVQADDVATYYCQQSYEDPWTFGGGTKLEIK | M14 and canine light chain |
| | RNDAQPAVYLFQPSPDQLHTGSASVVCLLNSFYPKDI | constant region |
| | NVKWKVDGVIQDTGIQESVTEQDKDSTYSLSSTLTMS | |
| | STEYLSHELYSCEITHKSLPSTLIKSFQRSECQRVD | |
| 22 | MLSHTGPSRFALFLLCSMETLLSSHMAPTHQLPPSDV | Canine IL31 amino acid |
| | RKIILELQPLSRGLLEDYQKKETGVPESNRILLLCLT | sequence |
| | SDSQPPRLNSSAILPYFRAIRPLSDKNIIDKIIEQLD | |
| | KLKFQHEPETEISVPADTFECKSFILTILQQFSACLE | |
| | SVFKSLNSGPQ | |
| 23 | PSDVRKIILELQPLSRG | Canine IL31 epitope |
| 24 | DIVLTQSPASLAVSLGQRATISCRASESVDTYGNSFM | Variable light chain amino |
| | HWYQQKSGQSPKLLIYRASNLESGIPARFGGSGSRTD | acid sequence of mouse |
| | FTLTIDPVEADDVATYYCQQSYEDPWTFGGGTKLEIK | antibody clone M14 |
| 25 | EVQLQESGPSLVKPSQTLSLTCSVTGDSITSGYWNWI | Variable heavy chain amino |
| | RKFPGNKLEYMGYISYSGITDYNPSLKSRISITRDTS | acid sequence of mouse |
| | KNQYYLQLNSVTTEDTATYYCARYGNYGYAMDYWGQG | antibody clone M14 |
| | TSVTVSS | |
| 26 | DIVLTQSPASLAVSLGQRATISCRASESVDTYGNSFM | Chimeric variable light chain |
| | HWYQQKSGQSPKLLIYRASNLESGIPARFGGSGSRTD | of mouse antibody clone M14 |
| | FTLTIDPVEADDVATYYCQQSYEDPWTFGGGTKLEIK | and canine light chain constant |
| | RNDAQPAVYLFQPSPDQLHTGSASVVCLLNSFYPKDI | region |
| | NVKWKVDGVIQDTGIQESVTEQDKDSTYSLSSTLTMS | |
| | STEYLSHELYSCEITHKSLPSTLIKSFQRSECQRVD | |
| 27 | EVQLQESGPSLVKPSQTLSLTCSVTGDSITSGYWNWI | Chimeric variable heavy chain |
| | RKFPGNKLEYMGYISYSGITDYNPSLKSRISITRDTS | of mouse antibody clone M14 |
| | KNQYYLQLNSVTTEDTATYYCARYGNYGYAMDYWGQG | and canine IgG-B |
| | TSVTVSSASTTAPSVFPLAPSCGSTSGSTVALACLVS | |
| | GYFPEPVTVSWNSGSLTSGVHTFPSVLQSSGLYSLSS | |
| | MVTVPSSRWPSETFTCNVAHPASKTKVDKPVPKRENG | |
| | RVPRPPDCPKCPAPEMLGGPSVFIFPPKPKDTLLIAR | |
| | TPEVTCVVVDLDPEDPEVQISWFVDGKQMQTAKTQPR | |
| | EEQFNGTYRVVSVLPIGHQDWLKGKQFTCKVNNKALP | |
| | SPIERTISKARGQAHQPSVYVLPPSREELSKNTVSLT | |
| | CLIKDFFPPDIDVEWQSNGQQEPESKYRTTPPQLDED | |
| | GSYFLYSKLSVDKSRWQRGDTFICAVMHEALHNHYTQ | |
| | ESLSHSPGK | |
| 28 | MLSHAGPARFALFLLCCMETLLPSHMAPAHRLQPSDV | Feline IL31 amino acid |
| | RKIILELRPMSKGLLQDYLKKEIGLPESNHSSLPCLS | sequence |
| | SDSQLPHINGSAILPYFRAIRPLSDKNTIDKIIEQLD | NCBI ref: XP_011286140.1 |
| | KLKFQREPEAKVSMPADNFERKNFILAVLQQFSACLE | [felis catus] |
| | HVLQSLNSGPQ | |
| 29 | MVSHIGSTRFALFLLCCLGTLMFSHTGPIYQLQPKEI | Equine IL31 amino acid |
| | QAIIVELQNLSKKLLDDYLNKEKGVQKFDSDLPSCFT | sequence |
| | SDSQAPGNINSSAILPYFKAISPSLNNDKSLYIIEQL | |
| | DKLNFQNAPETEVSMPTDNFERKRFILTILRWFSNCL | |
| | ELAMKTLTTAEQALPPLDPSTPHAGAVALTHHQQDRT | |
| | ALDRAVFPFVWAAPRGGEVGDGGH | |
| 30 | DIVLTQSPASLAVSLGQRATISCRASESVDTYGNSFM | Chimeric variable light chain |
| | HWYQQKSGQSPKLLIYRASNLESGIPARFGGSGSRTD | of mouse antibody clone M14 |
| | FTLTIDPVEADDVATYYCQQSYEDPWTFGGGTKLEIK | and feline light chain constant |
| | RSDAQPSVFLFQPSLDELHTGSASIVCILNDFYPKEV | region |
| | NVKWKVDGVVQNKGIQESTTEQNSKDSTYSLSSTLTM | |
| | SSTEYQSHEKFSCEVTHKSLASTLVKSFNRSECQRE | |
| 31 | EVQLQESGPSLVKPSQTLSLTCSVTGDSITSGYWNWI | Chimeric variable heavy chain |
| | RKFPGNKLEYMGYISYSGITDYNPSLKSRISITRDTS | of mouse antibody clone M14 |
| | KNQYYLQLNSVTTEDTATYYCARYGNYGYAMDYWGQG | and feline heavy chain |
| | TSVTVSSASTTAPSVFPLAPSCGTTSGATVALACLVL | constant region |
| | GYFPEPVTVSWNSGALTSGVHTFPAVLQASGLYSLSS | |
| | MVTVPSSRWLSDTFTCNVAHPPSNTKVDKTVRKTDHP | |
| | PGPKPCDCPKCPPPEMLGGPSIFIFPPKPKDTLSISR | |
| | TPEVICLVVDLGPDDSDVQITWFVDNIQVYTAKTSPR | |
| | EEQFNSTYRVVSVLPILHQDWLKGKEFKCKVNSKSLP | |
| | SPIERTISKAKGQPHEPQVYVLPPAQEELSRNKVSVT | |
| | CLIKSFHPPDIAVEWEITGQPEPENNYRTTPPQLDSD | |
| | GTYFVYSKLSVDRSHWQRGNTYTCSVSHEALHSHHTQ | |
| | KSLTQSPGK | |
| 32 | EIQMTQSPSSLSASPGDRVTISCRASESVDTYGNSFM | Felinized variable light chain |
| | HWYQQKPGQSPKLLIYRASNLESGVPSRFSGSGSGTD | sequence from mouse antibody |
| | FTLTISSLEPEDAATYYCQQSYEDPWTFGGGTKLEIK | clone M14 |
| 33 | DVQLVESGGDLVKPGGSLRLTCSVTGDSITSGYWNWV | Felinized variable heavy chain |
| | RQAPGKGLQWVAYISYSGITDYADSVKGRFTISRDNA | sequence from mouse antibody |
| | KNTLYLQLNNLKAEDTATYYCARYGNYGYAMDYWGQG | clone M14 |
| | TLVTVSS | |
| 79 | DVQLVESGGDLVKPGGSLRLTCSVT | Felinized M14 HC-FR1 |
| 80 | NWVRQAPGKGLQWVA | Felinized M14 HC-FR2 |
| 81 | ADSVKGRFTISRDNAKNTLYLQLNNLKAEDTATYYCA | Felinized M14 HC-FR3 |
| 82 | WGQGTLVTVSS | Felinized M14 HC-FR4 |
| 83 | EIQMTQSPSSLSASPGDRVTISC | Felinized M14 LC-FR1 |
| 84 | WYQQKPGQSPKLLIY | Felinized M14 LC-FR2 |
| 85 | GVPSRFSGSGSGTDFTLTISSLEPEDAATYYC | Felinized M14 LC-FR3 |
| 86 | FGGGTKLEIK | Felinized M14 LC-FR4 |
| 34 | EIQMTQSPSSLSASPGDRVTISCRASESVDTYGNSFM | Felinized light chain sequence |
| | HWYQQKPGQSPKLLIYRASNLESGVPSRFSGSGSGTD | from mouse antibody clone |
| | FTLTISSLEPEDAATYYCQQSYEDPWTFGGGTKLEIK | M14 |
| | RSDAQPSVFLFQPSLDELHTGSASIVCILNDFYPKEV | |
| | NVKWKVDGVVQNKGIQESTTEQNSKDSTYSLSSTLTM | |
| | SSTEYQSHEKFSCEVTHKSLASTLVKSFNRSECQRE | |
| 35 | DVQLVESGGDLVKPGGSLRLTCSVTGDSITSGYWNWV | Felinized heavy chain |
| | RQAPGKGLQWVAYISYSGITDYADSVKGRFTISRDNA | sequence from mouse antibody |
| | KNTLYLQLNNLKAEDTATYYCARYGNYGYAMDYWGQG | clone M14 |
| | TLVTVSSASTTAPSVFPLAPSCGTTSGATVALACLVL | |
| | GYFPEPVTVSWNSGALTSGVHTFPAVLQASGLYSLSS | |
| | MVTVPSSRWLSDTFTCNVAHPPSNTKVDKTVRKTDHP | |
| | PGPKPCDCPKCPPPEMLGGPSIFIFPPKPKDTLSISR | |
| | TPEVICLVVDLGPDDSDVQITWFVDNIQVYTAKTSPR | |
| | EEQFNSTYRVVSVLPILHQDWLKGKEFKCKVNSKSLP | |
| | SPIERTISKAKGQPHEPQVYVLPPAQEELSRNKVSVT | |
| | CLIKSFHPPDIAVEWEITGQPEPENNYRTTPPQLDSD | |
| | GTYFVYSKLSVDRSHWQRGNTYTCSVSHEALHSHHTQ | |
| | KSLTQSPGK | |
| 36 | METDTLLLWVLLLWVPGSTGDIVLTQSPASLAVSLGQ | Light chain amino acid |
| | RATISCRASESVDTYGNSFMHWYQQKSGQSPKLLIYR | sequence of mouse antibody |
| | ASNLESGIPARFGGSGSRTDFTLTIDPVEADDVATYY | clone M14 |
| | CQQSYEDPWTFGGGTKLEIKRADAAPTVSIFPPSSEQ | |
| | LTSGGASVVCFLNNFYPKDINVKWKIDGSERQNGVLN | |
| | SWTDQDSKDSTYSMSSTLTLIKDEYERHNSYTCEATH | |
| | KTSTSPIVKSFNRNEC | |
| 37 | METDTLLLWVLLLWVPGSTGDIVLTQSPASLAVSPGQ | Light chain amino acid |
| | RATISCRASESVDTYGNSFIHWYQQKPGQSPKLLIYR | sequence of mouse antibody |
| | ASNLESGIPARFSGSGSRTDFTLTINPVETDDVATYY | clone M18 |
| | CQQSYEDPWTFGGGTKLEIKRADAAPTVSIFPPSSEQ | |
| | LTSGGASVVCFLNNFYPKDINVKWKIDGSERQNGVLN | |
| | SWTDQDSKDSTYSMSSTLTLIKDEYERHNSYTCEATH | |
| | KTSTSPIVKSFNRNEC | |
| 38 | METDTLLLWVLLLWVPGSTGDIVLTQSPASLAVSLGQ | Light chain amino acid |
| | RATISCRASESVDTYGNSFMHWYQQKPGQPPKLLIYR | sequence of mouse antibody |
| | ASNLESGIPARFSGSGSRTDFTLTINPVEADDIATYY | clone M19 |
| | CQQSYEDPWTFGGGTKLEIKRADAAPTVSIFPPSSEQ | |
| | LTSGGASVVCFLNNFYPKDINVKWKIDGSERQNGVLN | |
| | SWTDQDSKDSTYSMSSTLTLIKDEYERHNSYTCEATH | |
| | KTSTSPIVKSFNRNEC | |
| 39 | METDTLLLWVLLLWVPGSTGDIVLTQSPASLAVSLGQ | Light chain amino acid |
| | RATISYRASKSVSTSGYSYMHWNQQKPGQPPRLLIYL | sequence of mouse antibody |
| | VSNLESGVPARFSGSGSGTDFTLNIHPVEEEDAATYY | clone M87 |
| | CQHIRELTRSFGGGTKLEIKRADAAPTVSIFPPSSEQ | |
| | LTSGGASVVCFLNNFYPKDINVKWKIDGSERQNGVLN | |
| | SWTDQDSKDSTYSMSSTLTLIKDEYERHNSYTCEATH | |
| | KTSTSPIVKSFNRNEC | |
| 64 | METDTLLLWVLLLWVPGSTGDIVLTQSPASLAVSPGQ | Variable light chain amino |
| | RATISCRASESVDTYGNSFIHWYQQKPGQSPKLLIYR | acid sequence of mouse |
| | ASNLESGIPARFSGSGSRTDFTLTINPVETDDVATYY | antibody clone M18 |
| | CQQSYEDPWTFGGGTKLEIK | |
| 65 | METDTLLLWVLLLWVPGSTGDIVLTQSPASLAVSLGQ | Variable light chain amino |
| | RATISCRASESVDTYGNSFMHWYQQKPGQPPKLLIYR | acid sequence of mouse |
| | ASNLESGIPARFSGSGSRTDFTLTINPVEADDIATYY | antibody clone M19 |
| | CQQSYEDPWTFGGGTKLEIK | |
| 66 | METDTLLLWVLLLWVPGSTGDIVLTQSPASLAVSLGQ | Variable light chain amino |
| | RATISYRASKSVSTSGYSYMHWNQQKPGQPPRLLIYL | acid sequence of mouse |
| | VSNLESGVPARFSGSGSGTDFTLNIHPVEEEDAATYY | antibody clone M87 |
| | CQHIRELTRSFGGGTKLEIK | |
| 40 | MAVLGLLLCLVTFPSCVLSEVQLQESGPSLVKPSQTL | Variable and hinge heavy |
| | SLTCSVTGDSITSGYWNWIRKFPGNKLEYMGYISYSG | chain amino acid sequence of |
| | ITDYNPSLKSRISITRDTSKNQYYLQLNSVTTEDTAT | mouse antibody clone M14 |
| | YYCARYGNYGYAMDYTNGQGTSVTVSSAKTTPPSVYPL | |
| | APGS | |
| 41 | MAVLGLLFCLVTFPSCVLSEVQLQESGPSLVKPSQTL | Variable and hinge heavy |
| | SLTCSVTGDSITSGYWNWIRKFPGNKLEYMGYISYSG | chain amino acid sequence of |
| | ITDYNPSLKSRISITRDTSKNQYYLQLNSVTTEDTAT | mouse antibody clone M18 |
| | YYCARYGNYGYAMDYTNGQGTSVTVSSAKTTPPSVYPL | |
| | APGS | |
| 42 | MAVLGLLFCLVTFPSCVLSEVQLQESGPSLVKPSQTL | Variable and hinge heavy |
| | SLTCSVTGDSITSGYWNWIRKFPGNELEYMGYISYSG | chain amino acid sequence of |
| | ITYYNPSLKSRFSITRDTSKNQYYLQLNSVTTEDTAT | mouse antibody clone M19 |
| | YYCARYGNYGYAMDYTNGQGTSVTVSSAKTTPPSVYPL | |
| | APGS | |
| 43 | MAVLGLLFCLVTFPSCVLSEVKLVESGGGLVQPGGSL | Variable and hinge heavy |
| | RLSCATSGFTFTDYYMNWVRQPPGKALETNLGFIRNKA | chain amino acid sequence of |
| | NGYTTEYSASVKGRFTISRDNSQSILYLQMNTLRAED | mouse antibody clone M87 |
| | SATYYCARDYYGSCFDYTNGQGTTLIVSSAKTTPPSVY | |
| | PLAPGS | |
| 67 | MAVLGLLFCLVTFPSCVLSEVQLQESGPSLVKPSQTL | Variable heavy chain amino |
| | SLTCSVTGDSITSGYTNNWIRKFPGNKLEYMGYISSG | acid sequence of mouse |
| | ITDYNPSLKSRISITRDTSKNQYYLQLNSVTTEDTAT | antibody clone M18 |
| | YYCARYGNYGYAMDYTNGQGTSVIVSS | |
| 68 | MAVLGLLFCLVTFPSCVLSEVQLQESGPSLVKPSQTL | Variable heavy chain amino |
| | SLTCSVTGDSITSGYTNNWIRKFPGNELEYMGYISYSG | acid sequence of mouse |
| | ITYYNPSLKSRFSITRDTSKNQYYLQLNSVTTEDTAT | antibody clone M19 |
| | YYCARYGNYGYAMDYTNGQGTSVIVSS | |
| 69 | MAVLGLLFCLVTFPSCVLSEVKLVESGGGLVQPGGSL | Variable heavy chain amino |
| | RLSCATSGFTFTDYYMNWVRQPPGKALETNLGFIRNKA | acid sequence of mouse |
| | NGYTTEYSASVKGRFTISRDNSQSILYLQMNTLRAED | antibody clone M87 |
| | SATYYCARDYYGSCFDYTNGQGTTLIVSS | |
| 44 | SSHMAPTHQLPPSDVRKIILELQPLSRGLLEDYQKKE | Mature canine IL31 amino |
| | TGVPESNRILLLCLTSDSQPPRLNSSAILPYFRAIRP | acid sequence |
| | LSDKNIIDKIIEQLDKLKFQHEPETEISVPADTFECK | |
| | SFILTILQQFSACLESVFKSLNSGPQ | |
| 45 | PSDX₁X₂KI, wherein X₁ and X₂ are any amino | IL31 epitope |
| | acids; or wherein X₁ is a hydrophobic | |
| | amino acid, or wherein X₁ is selected | |
| | from A, V, I, and L, or wherein X₁ is | |
| | selected from V and I; and wherein X₂ is a | |
| | hydrophilic amino acid, or wherein X₂ is | |
| | selected from A, R, K, Q, and N; or | |
| | wherein X₂ is selected from R and Q. | |
| 88 | PSDVRKI | Alternative canine IL31 |
| | | epitope |
| 46 | MASHSGPSTSVLFLFCCLGGWLASHTLPVRLLRPSDD | Human IL31 precursor amino |
| | VQKIVEELQSLSKMLLKDVEEEKGVLVSQNYTLPCLS | acid sequence |
| | PDAQPPNNIHSPAIRAYLKTIRQLDNKSVIDEIIEHL | NCBI ref: NP_001014358.1 |
| | DKLIFQDAPETNISVPTDTHECKRFILTISQQFSECM | |
| | DLALKSLTSGAQQATT | |
| 47 | MLSHAGPARFALFLLCFMGTSLTSQTAPIHQLHPSDV | Predicted walrus IL31 amino |
| | RKIILELQPLSKGLLEDYLKKEMGVPESNHFLLPCLT | acid sequence |
| | SDSQPPRINSSAILPYFRAIRPLSDKNTINKIIEQLD | NCBI ref: XP_004395998.1 |
| | KLKFQHEPETEVSVPADTFESKSFILAILQQFSACLD | [odobenus rosmarus divergens] |
| | HVFKSLNPGPQQVMQGHLIEPIPSGTADV | |
| 48 | MLSHAGPARFALFLLCFMGTSLTSQTAPIHQLHPSDV | Walrus_IL31_His6 |
| | RKIILELQPLSKGLLEDYLKKEMGVPESNHFLLPCLT | |
| | SDSQPPRINSSAILPYFRAIRPLSDKNTINKIIEQLD | |
| | KLKFQHEPETEVSVPADTFESKSFILAILQQFSACLD | |
| | HVFKSLNPGPQQVMQGHLIEPIPSGTADVGSGSHHHH | |
| | HH | |
| 49 | MASHSGPATSVLFLLCCLGGWLTSHTLPVHFLQPSDI | Predicted olive baboon IL31 |
| | QKIVEELQSLSKMLLKDVKEDKGVLVSQNYTLPCLTP | amino acid sequence |
| | DAQPPNIIHSPAIRAYLKTIRELDNKSVIDEIIEHLD | NCBI ref: XP_003907358.1 |
| | KLIFQDAPETNISVPTDTHECKRFILTISQQFSECMD | [papio anubis] |
| | LALKSLTSGAQQATT | |
| 50 | MASHSGPATSVLFLLCCLGGWLTSHTLPVHFLQPSDI | Olive baboon_IL31_His6 |
| | QKIVEELQSLSKMLLKDVKEDKGVLVSQNYTLPCLTP | |
| | DAQPPNIIHSPAIRAYLKTIRELDNKSVIDEIIEHLD | |
| | KLIFQDAPETNISVPTDTHECKRFILTISQQFSECMD | |
| | LALKSLTSGAQQATTGSGSHHHHHH | |
| 51 | MLSRAVPAGFALFLLCYMETLLTSHTAPTHRLPPSDI | Predicted polar bear IL31 |
| | RKIILELQPLSKGLLEDYLKKETGLPESNHSLVPCLT | amino acid sequence |
| | SDSEAPHINSSAILPYFRAIRPLSDKNVIDKIIEQLD | NCBI ref: XP_008687166.1 |
| | KLKFQHEPETEVSVPADTFEGKSFILTILQQFSACLE | [ursus mantimus] |
| | RVFKSLNPGAQ | |
| 52 | MLSHAGPARFALFLLCFMETSLTSQTVPIHQLQPSDV | Predicted weddell seal IL31 |
| | RKIILELQPLSKGLLEDYLKKEMGVPESNHFLLPCLT | amino acid sequence |
| | SDSQPPRINSSAILPYFRAIRPLSDKNIINKIIEQLD | NCBI ref: XP_006746595.1 |
| | KLKFQHEPETEVSVPADTFESKSFILTILQQFSACLG | [leptonychotes weddellii] |
| | HVLKSLNPGPQQVMQGHLAKPIPSGTADMYETLRHHH | |
| 53 | MLSHAGPARFALFLLCCMETLLPSHMAPTHRLPSDV | Predicted amur tiger IL31 |
| | RKIILELRPMSKGLLQDYLKKEMGLPESNHSSLPCLS | amino acid sequence |
| | SDSQLPHINGSAILPYFRAIRPLSDKNTIDKIIEQLD | NCBI ref: XP_007079636.1 |
| | KLKFQREPEAEVSMPADNFERKNFILAVLQQFSACLE | [panthera tigris altaica] |
| | HVLQSLNSGPQ | |
| 54 | MLSHAGPARFALFLLCCMETLLPSHMAPTHRLQPSDV | Predicted cheetah IL31 amino |
| | RKIILELRPMSKGLLQDYLKKEMGLPESNHSSLPCLS | acid sequence |
| | SDSQLPHINGSAILPYFRAIRPLSDKNTIDKIIEQLD | NCBI ref: XP_014919275.1 |
| | KLKFQREPEAEVSMPADNFERKNFILAVLQQFSACLE | [acinonyx jubatus] |
| | HVLQSLNSGSQ | |
| 55 | MASHSGPATSVLFLLCCLGGWLTSHTLPVHFLQPSDI | Predicted crab-eating macaque |
| | QKIVEELQSLSKMLLKDVKEDKGVLVSQNYTLPCLTP | IL31 amino acid sequence |
| | DAQPPNIIHSPAIRAYLKTIRQLDNKSVIDEIIEHLD | GenBank ref: EHH66805.1 |
| | KLIFQDAPETNISVPTDTHECKRFILTISQQFSECMD | [macaca fascicularis] |
| | LALKSLTSGAQQATT | |
| 56 | GPATSVLFLLCCLGGWLTSHTLPVHFLQPSDIQKIVE | Predicted rhesus monkey IL31 |
| | ELQSLSKMLLKDVKEDKGVLVSQNYTLPCLTPDAQPP | amino acid sequence (partial) |
| | NIIHSPAIRAYLKTIRQLDNKSVIDEIIEHLDKLIFQ | GenBank ref: EHH21279.1 |
| | DAPETNISVPTDTHECKRFILTISQQFSECMDLALKS | [macaca mulatta] |
| | LTSGAQQATT | |
| 57 | MASHSGPATSVLFLLCCLGGWLTSHTLPVHFLQPSDI | Predicted drill IL31 amino acid |
| | QKIVKELQSLSKMLLKDVKEDKGVLVSQNYTLPCLTP | sequence |
| | DAQPPNIIHSPAIRAYLKTIRQLDNRSVIDEIIEHLD | NCBI ref: XP_011819882.1 |
| | KLIFQDAPETNISVPTDTHECKRFILTISQQFSECMD | [mandrillus leucophaeus] |
| | LALKSLTSGAQQATT | |
| 58 | MASHSGPATSVLFLLCCLGGWLTSHTLPVHFLQPSDI | Predicted green monkey IL31 |
| | QKIVEELQSLSKMLLKDVKEDKGVLVSQNYKLPCLTP | amino acid sequence |
| | DAQPPNIIHSPAIRAYLKTIRQLDNKSVIDEIIEHLD | NCBI ref: XP_008003211.1 |
| | KLIFQDAPETNISVPTDTHECKRFILTISQQFSECMD | [chlorocebus sabaeus] |
| | LALKSLTSGAQQATT | |
| 59 | MASHSGPATSVLFLLCCLGGWLTSHTLPVHFLQPSDI | Predicted sooty mangebey |
| | QKIVEELQSLSKMLLKDVKEDKGVLVSQNYTLPCLTP | IL31 amino acid sequence |
| | DAQPPNIIHSPAIRAYLKTIRQLDNRSVIDEIIEHLD | NCBI ref: XP_011926625.1 |
| | KLIFQDAPETNISVPTDTHECKRFILTISQQFSECMD | [cercocebus atys] |
| | LALKSLTSGAQQATT | |
| 60 | MASHSGPTTSVLFLLCCLGGWLTSHTLPVHFLRPSDI | Predicted golden snub-nosed |
| | QKIVEELQSLSKMLLKDVEEDKGVLVSQNYTLPCLTP | monkey IL31 amino acid |
| | DAQPPNIIHSPAIRAYLKTIRQLDNKSVIDEIIEHLD | sequence |
| | KLIFQDAPETNISVPTDTHECKRFILTISQQFSECMD | NCBI ref: XP_010366647.1 |
| | LALKSLTSGAQQATT | [rhinopithecus roxellana] |
| 61 | MIFHTGTTKPTLVLLCCIGTWLATCSLSFGAPISKED | Murine IL31 precursor amino |
| | LRTTIDLLKQESQDLYNNYSIKQASGMSADESIQLPC | acid sequence |
| | FSLDREALTNISVIIAHLEKVKVLSENTVDTSWVIRW | NCBI ref: NP_083870 |
| | LTNISCFNPLNLNISVPGNTDESYDCKVFVLTVLKQF | [mus musculus] |
| | SNCMAELQAKDNTTC | |

### DESCRIPTION OF CERTAIN EMBODIMENTS

Antibodies that bind canine IL31, feline IL31, or equine IL31 are provided. Antibody heavy chains and light chains that are capable of forming antibodies that bind IL31 are also provided. In addition, antibodies, heavy chains, and light chains comprising one or more particular complementary determining regions (CDRs) are provided. Polynucleotides encoding antibodies to canine IL31 are provided. Methods of producing or purifying antibodies to canine IL31 are also provided. Methods of treatment using antibodies to canine IL31 are provided. Such methods include, but are not limited to, methods of treating IL31-induced conditions in companion animal species. Methods of detecting IL31 in a sample from a companion animal species are provided.

For the convenience of the reader, the following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context ofa multiple dependent claim, the use of “or” when referring back to other claims refers to those claims in the alternative only.

Anti-IL31 Antibodies

Novel antibodies directed against IL31 are provided, for example antibodies that bind to canine IL31, feline IL31, and/or equine IL31. Anti-IL31 antibodies provided herein include, but are not limited to, monoclonal antibodies, mouse antibodies, chimeric antibodies, caninized antibodies, felinized antibodies, and equinized antibodies. In some embodiments, an anti-IL31 antibody is an isolated mouse monoclonal antibody such as M14, M18, M19, and M87.

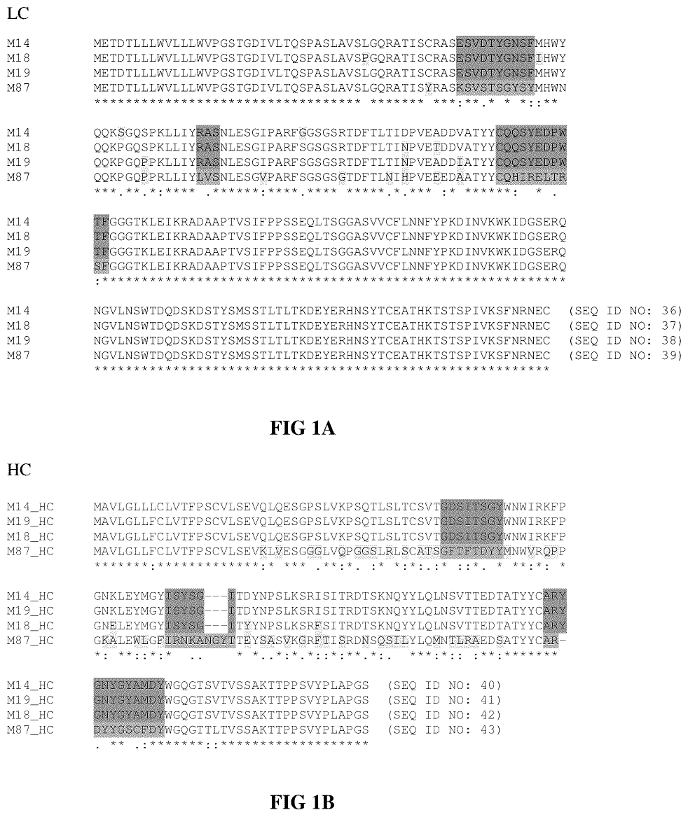

Monoclonal antibodies M14, M18, M19, and M87 were isolated as follows. Briefly, mice were immunized with canine IL31 and mouse monoclonal antibody clones were obtained through standard hybridoma technology. Enzyme linked immunosorbent assay (ELISA) was used to screen for hybridoma clones producing IL31-binding antibodies. Based on binding affinity and a cell-based functional assay described herein, hybridoma clones producing monoclonal antibodies M14, M18, M19, and M87 were selected for further investigation. The variable heavy chain (VH) and variable light chain (VL) of each of the four clones were sequenced and analyzed by sequence alignment (FIG. 1).

Also provided herein are amino acid sequences of monoclonal antibody M14. For example, the variable heavy chain CDRs (SEQ ID NOs: 1-3; SEQ ID NO: 89 is an alternate definition for CDR-H2), variable light chain CDRs (SEQ ID NOs: 8-10), variable region heavy chain framework sequences (SEQ ID NOs: 4-7), and variable region light chain framework sequences (SEQ ID NOs: 11-14) for monoclonal antibody M14 are provided. The amino acid sequences of the variable light chain, light chain, variable heavy chain, and variable and hinge heavy chain of monoclonal antibody M14 are provided (SEQ ID NOs: 24, 36, 25, and 40, respectively).

In addition, the amino acid sequences of the CDRs, framework sequences, variable light chain, variable heavy chain of monoclonal antibodies M18, M19, and M87 are provided (See, e.g., FIG. 1). The variable heavy chain CDRs (SEQ ID NOs: 1-3; SEQ ID NO: 89 is an alternate definition for CDR-H2), variable light chain CDRs (SEQ ID NOs: 8-10), variable light chain (SEQ ID NO: 65), light chain (SEQ ID NO: 38), variable and hinge heavy chain (SEQ ID NO: 42), and variable heavy chain (SEQ ID NO: 68) for monoclonal antibody M19 are provided. The variable heavy chain CDRs (SEQ ID NOs: 1, 62, and 3; SEQ ID NO: 87 is an alternate definition for CDR-H2), variable light chain CDRs (SEQ ID NOs: 63, 9, and 10), variable light chain (SEQ ID NO: 64), light chain (SEQ ID NO: 37), variable and hinge heavy chain (SEQ ID NO: 41), and variable heavy chain (SEQ ID NO: 67) for monoclonal antibody M18 are provided. The variable light chain (SEQ ID NO: 66), light chain (SEQ ID NO: 39), variable and hinge heavy chain (SEQ ID NO: 43), and variable heavy chain (SEQ ID NO: 69) for monoclonal antibody M87 are provided.

Also provided herein are chimeric, caninized, felinized, and equinized antibodies derived from monoclonal antibody M14, M18, M19, and M87. In some embodiments, amino acid sequences of caninized monoclonal antibody M14 are provided, such as SEQ ID NOs: 15-21 and 70-78. In some embodiments, amino acid sequences of felinized antibodies derived from monoclonal antibody M14 are provided, such as SEQ ID NOs: 32-35 and 79-86. In some embodiments, amino acid sequences of chimeric antibodies derived from monoclonal antibody M14 are provided, such as SEQ ID NOs: 26, 27, 30, and 31.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′)₂, ScFv, minibody, diabody, triabody, and tetrabody) so long as they exhibit the desired antigen-binding activity. Canine, feline, and equine species have different varieties (classes) of antibodies that are shared by many mammalians.

The term antibody includes, but is not limited to, fragments that are capable of binding to an antigen, such as Fv, single-chain Fv (scFv), Fab, Fab′, di-scFv, sdAb (single domain antibody) and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Thus, if a murine version of an antibody is disclosed, one of skill in the art will appreciate how to transform the murine sequence based antibody into a cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y ⁹⁹Tc, ¹¹¹In, ¹²⁵I, ¹³¹I, ¹⁷⁷, Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to poly clonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

In some embodiments, the monoclonal antibody is an isolated mouse antibody selected from clone M14, M18, M19, and M87.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“IL31” as used herein refers to any native IL31 that results from expression and processing of IL31 in a cell. The term includes IL31 from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL31, e.g., splice variants or allelic variants.

In some embodiments, a canine IL31 comprises the amino acid sequence of SEQ ID NO: 22 or SEQ ID NO: 44. In some embodiments, a feline IL31 comprises the amino acid sequence of SEQ ID NO: 28. In some embodiments, an equine IL31 comprises the amino acid sequence of SEQ ID NO: 29. In some embodiments, a human IL31 comprises the amino acid sequence of SEQ ID NO: 46. In some embodiments, a walrus IL31 comprises the amino acid sequence of SEQ ID NO: 47. In some embodiments, a murine IL31 comprises the amino acid sequence of SEQ ID NO: 61. In other embodiments, the IL31 comprises the amino acid sequence of SEQ ID NO: 49, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or SEQ ID NO: 60.

The term “IL31 binding domain” of an antibody means the binding domain formed by a light chain and heavy chain of an anti-IL31 antibody, which binds IL31.

In some embodiments, the IL31 binding domain binds canine IL31 with greater affinity than it binds human IL31. In some embodiments, the IL31 binding domain binds IL31 of other companion animals, such as feline IL31 or equine IL31. In some embodiments, the IL31 binding domain does not bind human IL31.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

In some embodiments, the epitope comprises the amino acid sequence PSDX₁×₂KI (SEQ ID NO: 45), wherein X is any amino acid residue. In some embodiments, X₁ is a hydrophobic amino acid. In some embodiments, X₁ is selected from A, V, I, and L. In some embodiments, X₁ is selected from V and I. In some embodiments, X₂ is a hydrophilic amino acid. In some embodiments, X₂ is selected from A, R, K, Q, and N. In some embodiments, X₂ is selected from R and Q. In some embodiments, X₁ is V and X₂ is R. In some embodiments, X₁ is I and X₂ is Q. In some embodiments, the epitope comprises the amino acid sequence of SEQ ID NO: 88. In some embodiments, the epitope comprises the amino acid sequence of SEQ ID NO: 23. In some embodiments, the epitope is within amino acids 34-50 of SEQ ID NO: 22. In some embodiments, the epitope comprises amino acids 34-50 of SEQ ID NO: 22.

The term “CDR” means a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, or a combination of the Kabat, Chothia, AbM, or contact definitions. The various CDRs within an antibody can be designated by their appropriate number and chain type, including, without limitation as CDR-H1, CDR-H2, CDR-H3, CDR-L1, CDR-L2, and CDR-L3. The term “CDR” is used herein to also encompass a “hypervariable region” or HVR, including hypervariable loops.

In some embodiments, an anti-IL31 antibody comprises a heavy chain comprising (a) a CDR-H1 comprising the amino acid sequence of SEQ ID NO: 1; (b) a CDR-H2 comprising the amino acid sequence of SEQ ID NO: 2, SEQ ID NO: 62, SEQ ID NO: 89, or SEQ ID NO: 87; or (c) a CDR-H3 comprising the amino acid sequence of SEQ ID NO: 3. In some embodiments, an anti-IL31 antibody comprises a light chain comprising (a) a CDR-L1 comprising the amino acid sequence of SEQ ID NO: 8 or SEQ ID NO: 63; (b) a CDR-L2 comprising the amino acid sequence of SEQ ID NO: 9; or (c) a CDR-L3 comprising the amino acid sequence of SEQ ID NO: 10.

In some embodiments, an anti-IL31 antibody comprises a heavy chain comprising (a) a CDR-H1 comprising the amino acid sequence of SEQ ID NO: 1, (b) a CDR-H2 comprising the amino acid sequence of SEQ ID NO: 2 or 89, and (c) a CDR-H3 comprising the amino acid sequence of SEQ ID NO: 3; and a light chain comprising (a) a CDR-L1 comprising the amino acid sequence of SEQ ID NO: 8 or 63, (b) a CDR-L2 comprising the amino acid sequence of SEQ ID NO: 9, and (c) a CDR-L3 comprising the amino acid sequence of SEQ ID NO: 10.

In some embodiments, an anti-IL31 antibody comprises a heavy chain comprising (a) a CDR-H1 comprising the amino acid sequence of SEQ ID NO: 1, (b) a CDR-H2 comprising the amino acid sequence of SEQ ID NO: 62 or 87, and (c) a CDR-H3 comprising the amino acid sequence of SEQ ID NO: 3; and a light chain comprising (a) a CDR-L1 comprising the amino acid sequence of SEQ ID NO: 8 or 63, (b) a CDR-L2 comprising the amino acid sequence of SEQ ID NO: 9, and (c) a CDR-L3 comprising the amino acid sequence of SEQ ID NO: 10.

The term “variable region” as used herein refers to a region comprising at least three CDRs. In some embodiments, the variable region includes the three CDRs and at least one framework region (“FR”). The terms “heavy chain variable region” or “variable heavy chain” are used interchangeably to refer to a region comprising at least three heavy chain CDRs. The terms “light chain variable region” or “variable light chain” are used interchangeably to refer to a region comprising at least three light chain CDRs. In some embodiments, the variable heavy chain or variable light chain comprises at least one framework region. In some embodiments, an antibody comprises at least one heavy chain framework region selected from HC-FR1, HC-FR2, HC-FR3, and HC-FR4. In some embodiments, an antibody comprises at least one light chain framework region selected from LC-FR1, LC-FR2, LC-FR3, and LC-FR4. The framework regions may be juxtaposed between light chain CDRs or between heavy chain CDRs. For example, an antibody may comprise a variable heavy chain having the following structure: (HC-FR1)-(CDR-H1)-(HC-FR2)-(CDR-H2)-(HC-FR3)-(CDR-H3)-(HC-FR4). An antibody may comprise a variable heavy chain having the following structure: (CDR-H1)-(HC-FR2)-(CDR-H2)-(HC-FR3)-(CDR-H3). An antibody may also comprise a variable light chain having the following structure: (LC-FR1)-(CDR-L1)-(LC-FR2)-(CDR-L2)-(LC-FR3)-(CDR-L3)-(LC-FR4). An antibody may also comprise a variable light chain having the following structure: (CDR-L1)-(LC-FR2)-(CDR-L2)-(LC-FR3)-(CDR-L3).

In some embodiments, an anti-IL31 antibody comprises one or more of (a) a variable region heavy chain framework 1 (HC-FR1) sequence of SEQ ID NO: 4, (b) a HC-FR2 sequence of SEQ ID NO: 5, (c) a HC-FR3 sequence of SEQ ID NO: 6, (d) a HC-FR4 sequence of SEQ ID NO: 7, (e) a variable region light chain framework 1 (LC-FR1) sequence of SEQ ID NO: 11, (f) an LC-FR2 sequence of SEQ ID NO: 12, (g) an LC-FR3 sequence of SEQ ID NO: 13, or (h) an LC-FR4 sequence of SEQ ID NO: 14. In some embodiments, an anti-IL31 antibody comprises a variable light chain sequence of (a) SEQ ID NO: 16, (b) SEQ ID NO: 24, or (c) SEQ ID NO: 32. In some embodiments, an anti-IL31 antibody comprises a variable heavy chain sequence of (a) SEQ ID NO: 15; (b) SEQ ID NO: 25; or (c) SEQ ID NO: 33. In some embodiments, an anti-IL31 antibody comprises (a) a variable light chain sequence of SEQ ID NO: 16 and a variable heavy chain sequence of SEQ ID NO: 15; (b) a variable light chain sequence of SEQ ID NO: 24 and a variable heavy chain sequence of SEQ ID NO: 25; or (c) a variable light chain sequence of SEQ ID NO: 32 and a variable heavy chain sequence of SEQ ID NO: 33.

The term “constant region” as used herein refers to a region comprising at least three constant domains. The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a 6 constant region is an IgD antibody, an antibody comprising an a constant region is an IgA antibody, an antibody comprising a constant region is an IgM antibody, and an antibody comprising an ε constant region is an IgE antibody. Certain isotypes can be further subdivided into subclasses. For example, IgG antibodies include, but are not limited to, IgG1 (comprising a 71 constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an at constant region) and IgA2 (comprising an a₂ constant region) antibodies; and IgM antibodies include, but are not limited to IgM1 and IgM2. The terms “light chain constant region” or “constant light chain” are used interchangeably to refer to a region comprising a light chain constant domain, CL. Nonlimiting exemplary light chain constant regions include λ and κ. Non-function-altering deletions and alterations within the domains are encompassed within the scope of the term “constant region” unless designated otherwise. Canine, feline, and equine have antibody classes such as IgG, IgA, IgD, IgE, and IgM. Within the canine IgG antibody class are IgG-A, IgG-B, IgG-C, and IgG-D. Within the feline IgG antibody class are IgG1a, IgG1b, and IgG2. Within the equine IgG antibody class are IgG1, IgG2, IgG3, IgG4, IgG5, IgG6, and IgG7.

The term “chimeric antibody” or “chimeric” refers to an antibody in which a portion of the heavy chain or light chain is derived from a particular source or species, while at least a part of the remainder of the heavy chain or light chain is derived from a different source or species. In some embodiments, a chimeric antibody refers to an antibody comprising at least one variable region from a first species (such as mouse, rat, cynomolgus monkey, etc.) and at least one constant region from a second species (such as human, dog, cat, equine, etc.). In some embodiments, a chimeric antibody comprises at least one mouse variable region and at least one canine constant region. In some embodiments, a chimeric antibody comprises at least one mouse variable region and at least one feline constant region. In some embodiments, all of the variable regions of a chimeric antibody are from a first species and all of the constant regions of the chimeric antibody are from a second species. In some embodiments, a chimeric antibody comprises a constant heavy chain region or constant light chain region from a companion animal. In some embodiments, a chimeric antibody comprises a mouse variable heavy and light chains and a companion animal constant heavy and light chains. For example, a chimeric antibody may comprise a mouse variable heavy and light chains and a canine constant heavy and light chains; a chimeric antibody may comprise a mouse variable heavy and light chains and a feline constant heavy and light chains; or a chimeric antibody may comprise a mouse variable heavy and light chains and an equine constant heavy and light chains.

In some embodiments, an anti-IL31 antibody comprises a chimeric antibody comprising:
a. (i) a light chain amino acid sequence of SEQ ID NO: 26; (ii) a heavy chain amino acid sequence of SEQ ID NO: 27; or (iii) a light chain amino acid sequence as in (i) and a heavy chain sequence as in (ii); or
b. (i) a light chain amino acid sequence of SEQ ID NO: 30; (ii) a heavy chain amino acid sequence of SEQ ID NO: 31; or (iii) a light chain amino acid sequence as in (i) and a heavy chain sequence as in (ii).

A “canine chimeric” or “canine chimeric antibody” refers to a chimeric antibody having at least a portion of a heavy chain or a portion of a light chain derived from a dog. A “feline chimeric” or “feline chimeric antibody” refers to a chimeric antibody having at least a portion of a heavy chain or a portion of a light chain derived from a cat. An “equine chimeric” or “equine chimeric antibody” refers to a chimeric antibody having at least a portion of a heavy chain or a portion of a light chain derived from a horse. In some embodiments, a canine chimeric antibody comprises a mouse variable heavy and light chains and a canine constant heavy and light chains. In some embodiments, a feline chimeric antibody comprises a mouse variable heavy and light chains and a feline constant heavy and light chains. In some embodiments, an equine chimeric antibody comprises a mouse variable heavy and light chains and an equine constant heavy and light chains. In some embodiments, the antibody is a chimeric antibody comprising murine variable heavy chain framework regions or murine variable light chain framework regions.

A “canine antibody” as used herein encompasses antibodies produced in a canine; antibodies produced in non-canine animals that comprise canine immunoglobulin genes or comprise canine immunoglobulin peptides; or antibodies selected using in vitro methods, such as phage display, wherein the antibody repertoire is based on a canine immunoglobulin sequence. The term “canine antibody” denotes the genus of sequences that are canine sequences. Thus, the term is not designating the process by which the antibody was created, but the genus of sequences that are relevant.

In some embodiments, an anti-IL31 antibody comprises a canine heavy chain constant region selected from an IgG-A, IgG-B, IgG-C, and IgG-D constant region. In some embodiments, an anti-IL31 antibody is a canine IgG-A, IgG-B, IgG-C, or IgG-D antibody. In some embodiments, an anti-IL31 antibody is (a) a canine IgG-A antibody comprising the heavy chain amino acid sequence of SEQ ID NO: 17; (b) a canine IgG-B antibody comprising the heavy chain amino acid sequence of SEQ ID NO: 18; (c) a canine IgG-C antibody comprising the heavy chain amino acid sequence of SEQ ID NO: 19; or (d) a canine IgG-D antibody comprising the heavy chain amino acid sequence of SEQ ID NO: 20.

A “feline antibody” as used herein encompasses antibodies produced in a feline; antibodies produced in non-feline animals that comprise feline immunoglobulin genes or comprise feline immunoglobulin peptides; or antibodies selected using in vitro methods, such as phage display, wherein the antibody repertoire is based on a feline immunoglobulin sequence. The term “feline antibody” denotes the genus of sequences that are feline sequences. Thus, the term is not designating the process by which the antibody was created, but the genus of sequences that are relevant.

In some embodiments, an anti-IL31 antibody comprises a feline heavy chain constant region selected from an IgG1, IgG2a, and IgG2b constant region. In some embodiments, an anti-IL31 antibody is a feline IgG1, IgG2a, or IgG2b antibody.

An “equine antibody” as used herein encompasses antibodies produced in an equine; antibodies produced in non-equine animals that comprise equine immunoglobulin genes or comprise equine immunoglobulin peptides; or antibodies selected using in vitro methods, such as phage display, wherein the antibody repertoire is based on an equine immunoglobulin sequence. The term “equine antibody” denotes the genus of sequences that are equine sequences. Thus, the term is not designating the process by which the antibody was created, but the genus of sequences that are relevant.

In some embodiments, an anti-IL31 antibody comprises an equine heavy chain constant region selected from an IgG1, IgG2, IgG3, IgG4, IgG5, IgG6 and IgG7 constant region. In some embodiments, an anti-IL31 antibody is an equine IgG1, IgG2, IgG3, IgG4, IgG5, IgG6 and IgG7 antibody.

A “caninized antibody” means an antibody in which at least one amino acid in a portion of a non-canine variable region has been replaced with the corresponding amino acid from a canine variable region. In some embodiments, a caninized antibody comprises at least one canine constant region (e.g., a γ constant region, an a constant region, a 6 constant region, an c constant region, a constant region, or etc.) or fragment thereof. In some embodiments, a caninized antibody is an antibody fragment, such as Fab, scFv, (Fab′)₂, etc. The term “caninized” also denotes forms of non-canine (for example, murine) antibodies that are chimeric immunoglobulins, immunoglobulin chains, or fragments thereof (such as Fv, Fab, Fab′, F(ab′)₂ or other antigen-binding sequences of antibodies) that contain minimal sequence of non-canine immunoglobulin. Caninized antibodies can include canine immunoglobulins (recipient antibody) in which residues from a CDR of the recipient are substituted by residues from a CDR of a non-canine species (donor antibody) such as mouse, rat, or rabbit having the desired specificity, affinity, and capacity. In some instances, Fv framework region (FR) residues of the canine immunoglobulin are replaced by corresponding non-canine residues. Furthermore, the caninized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a canine variable region. In some embodiments, the modified chain is fused to a canine constant heavy chain or a canine constant light chain. In some embodiments, an anti-IL31 antibody is a caninized antibody comprising (a) a heavy chain sequence of SEQ ID NO: 15, (b) a heavy chain sequence of SEQ ID NO: 17, (c) a heavy chain sequence of SEQ ID NO: 18, (d) a heavy chain sequence of SEQ ID NO: 19, (e) a heavy chain sequence of SEQ ID NO: 20, (f) a light chain sequence of SEQ ID NO: 16, or (g) a light chain sequence of SEQ ID NO: 21.

A “felinized antibody” means an antibody in which at least one amino acid in a portion of a non-feline variable region has been replaced with the corresponding amino acid from a feline variable region. In some embodiments, a felinized antibody comprises at least one feline constant region (e.g., a γ constant region, an a constant region, a 6 constant region, an c constant region, a constant region, or etc.) or fragment thereof. In some embodiments, a felinized antibody is an antibody fragment, such as Fab, scFv, (Fab′)₂, etc. The term “felinized” also denotes forms of non-feline (for example, murine) antibodies that are chimeric immunoglobulins, immunoglobulin chains, or fragments thereof (such as Fv, Fab, Fab′, F(ab′)₂ or other antigen-binding sequences of antibodies) that contain minimal sequence of non-feline immunoglobulin. Felinized antibodies can include feline immunoglobulins (recipient antibody) in which residues from a CDR of the recipient are substituted by residues from a CDR of a non-feline species (donor antibody) such as mouse, rat, or rabbit having the desired specificity, affinity, and capacity. In some instances, Fv framework region (FR) residues of the feline immunoglobulin are replaced by corresponding non-feline residues. Furthermore, the felinized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a feline variable region. In some embodiments, the modified chain is fused to a feline constant heavy chain or a canine constant light chain. In some embodiments, an anti-IL31 antibody is a felinized antibody comprising (a) a light chain sequence of SEQ ID NO: 32, (b) a light chain sequence of SEQ ID NO: 34, (c) a heavy chain sequence of SEQ ID NO: 33, or (d) a heavy chain sequence of SEQ ID NO: 35.

An “equinized antibody” means an antibody in which at least one amino acid in a portion of a non-equine variable region has been replaced with the corresponding amino acid from an equine variable region. In some embodiments, an equinized antibody comprises at least one equine constant region (e.g., a γ constant region, an a constant region, a 6 constant region, an c constant region, a constant region, or etc.) or fragment thereof. In some embodiments, an equinized antibody is an antibody fragment, such as Fab, scFv, (Fab′)₂, etc. The term “equinized” also denotes forms of non-equine (for example, murine) antibodies that are chimeric immunoglobulins, immunoglobulin chains, or fragments thereof (such as Fv, Fab, Fab′, F(ab′)₂ or other antigen-binding sequences of antibodies) that contain minimal sequence of non-equine immunoglobulin. Equinized antibodies can include equine immunoglobulins (recipient antibody) in which residues from a CDR of the recipient are substituted by residues from a CDR of a non-equine species (donor antibody) such as mouse, rat, or rabbit having the desired specificity, affinity, and capacity. In some instances, Fv framework region (FR) residues of the equine immunoglobulin are replaced by corresponding non-equine residues. Furthermore, the equinized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from an equine variable region. In some embodiments, the modified chain is fused to an equine constant heavy chain or a canine constant light chain.

The term “IgX Fc” means the Fc region is derived from a particular antibody isotype (e.g., IgG, IgA, IgD, IgE, IgM, etc.), where “X” denotes the antibody isotype. Thus, “IgG Fc” denotes the Fc region of a 7 chain, “IgA Fc” denotes the Fc region of an a chain, “IgD Fc” denotes the Fc region of a 6 chain, “IgE Fc” denotes the Fc region of an F chain, “IgM Fc” denotes the Fc region of a μ chain, etc. In some embodiments, the IgG Fc region comprises CH1, hinge, CH2, CH3, and CL1. “IgX—N-Fc” denotes that the Fc region is derived from a particular subclass of antibody isotype (such as canine IgG subclass A, B, C, or D; feline IgG subclass 1, 2a, or 2b; or equine IgG subclass IgG1, IgG2, IgG3, IgG4, IgG5, IgG6, or IgG7, etc.), where “N” denotes the subclass. In some embodiments, IgX Fc or IgX—N-Fc regions are derived from a companion animal, such as a dog, a cat, or a horse. In some embodiments, IgG Fc regions are isolated from canine γ heavy chains, such as IgG-A, IgG-B, IgG-C, or IgG-D. In some instances, IgG Fc regions are isolated from feline 7 heavy chains, such as IgG1, IgG2a, or IgG2b. Antibodies comprising an Fc region of IgG-A, IgG-B, IgG-C, or IgG-D may provide for higher expression levels in recombination production systems.

The term “affinity” means the strength of the sum total of noncovalent interactions between a single binding site of a molecule (for example, an antibody) and its binding partner (for example, an antigen). The affinity of a molecule X for its partner Y can generally be represented by the dissociation constant (K D). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of an antibody-antigen interaction. In some embodiments, the K_{d} of the antibody is measured by using biolayer interferometry assays using a biosensor, such as an Octet® System (Pall ForteBio LLC, Fremont, Calif) according to the supplier's instructions. Briefly, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K d) is calculated as the ratio of k off/k on. The term “k on” refers to the rate constant for association of an antibody to an antigen and the term “koff” refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA K_{D}, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

In some embodiments, an anti-IL31 antibody binds to canine IL31, feline IL31, or equine IL31 with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry. In some embodiments, an anti-IL31 antibody binds to canine IL31, feline IL31, or equine IL31 with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷ M, between 1×10⁻⁶ M and 5×10⁻⁸ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷ M, between 5×10⁻⁷ M and 5×10⁻⁸ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry. In some embodiments, an anti-IL31 antibody binds to canine IL31, feline IL31, or equine IL31, as determined by immunoblot analysis.

In some embodiments, an anti-IL31 antibody does not bind to human IL31 as determined by immunoblot analysis and/or biolayer interferometry.

In some embodiments, an anti-IL31 antibody is provided that competes with an anti-IL31 antibody described herein (such as M14, M18, M19, or M87) for binding to IL31. In some embodiments, an antibody that competes with binding with any of the antibodies provided herein can be made or used. In some embodiments, an anti-IL31 antibody is provided that competes with monoclonal M14 antibody in binding to canine IL31 or feline IL31.

A “variant” means a biologically active polypeptide having at least about 50% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide.

In some embodiments, a variant has at least about 50% amino acid sequence identity, at least about 60% amino acid sequence identity, at least about 65% amino acid sequence identity, at least about 70% amino acid sequence identity, at least about 75% amino acid sequence identity, at least about 80% amino acid sequence identity, at least about 85% amino acid sequence identity, at least about 90% amino acid sequence identity, at least about 95% amino acid sequence identity with the native sequence polypeptide.

As used herein, “percent (%) amino acid sequence identity” and “homology” with respect to a peptide, polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

An amino acid substitution may include but is not limited to the replacement of one amino acid in a polypeptide with another amino acid. Exemplary substitutions are shown in Table 2. Amino acid substitutions may be introduced into an antibody of interest and the products screened for a desired activity, for example, retained/improved antigen binding, decreased immunogenicity, or improved ADCC or CDC.

TABLE 2

| Original | |
|---|---|
| Residue | Exemplary Substitutions |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to common side-chain properties:
(1) hydrophobic: Norleucine, Met, Ala, Val, Leu, le;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

In some embodiments, an anti-IL31 antibody comprises a heavy chain and a light chain, wherein:
a. the heavy chain comprises a CDR-H1 sequence having at least 85% sequence identity, at least 90% sequence identity, at least 95% sequence identity, or at least 98% sequence identity to the amino acid sequence of SEQ ID NO: 1; a CDR-H2 sequence having at least 85% sequence identity, at least 90% sequence identity, at least 95% sequence identity, or at least 98% sequence identity to the amino acid sequence of SEQ ID NO: 2, SEQ ID NO: 62, SEQ ID NO: 89, or SEQ ID NO: 87; and a CDR-H3 sequence having at least 85% sequence identity, at least 90% sequence identity, at least 95% sequence identity, or at least 98% sequence identity to the amino acid sequence of SEQ ID NO: 3, and
b. the light chain comprises a CDR-L1 sequence having at least 85% sequence identity, at least 90% sequence identity, at least 95% sequence identity, or at least 98% sequence identity to the amino acid sequence of SEQ ID NO: 8 or SEQ ID NO: 63; a CDR-L2 sequence having at least 85% sequence identity, at least 90% sequence identity, at least 95% sequence identity, or at least 98% sequence identity to the amino acid sequence of SEQ ID NO: 9; and a CDR-L3 sequence having at least 85% sequence identity, at least 90% sequence identity, at least 95% sequence identity, or at least 98% sequence identity to the amino acid sequence of SEQ ID NO: 10.

In some embodiments, an anti-IL31 antibody comprises a heavy chain and a light chain, wherein:
a. (i) a variable light chain sequence having at least 85%, at least 90%, at least 95%, or at least 98% sequence identity to the amino acid sequence of SEQ ID NO: 24; (ii) a variable heavy chain sequence having at least 85%, at least 90%, at least 95%, or at least 98% sequence identity to the amino acid sequence of SEQ ID NO: 25; or (iii) a variable light chain sequence as in (i) and a variable heavy chain sequence as in (ii); or
b. (i) a variable light chain sequence having at least 85%, at least 90%, at least 95%, or at least 98% sequence identity to the amino acid sequence of SEQ ID NO: 16; (ii) a variable heavy chain sequence having at least 85%, at least 90%, at least 95%, or at least 98% sequence identity to the amino acid sequence of SEQ ID NO: 15; or (iii) a variable light chain sequence as in (i) and a variable heavy chain sequence as in (ii); or
c. (i) a variable light chain sequence having at least 85%, at least 90%, at least 95%, or at least 98% sequence identity to the amino acid sequence of SEQ ID NO: 32; (ii) a variable heavy chain sequence having at least 85%, at least 90%, at least 95%, or at least 98% sequence identity to the amino acid sequence of SEQ ID NO: 33; or (iii) a variable light chain sequence as in (i) and a variable heavy chain sequence as in (ii); or
d. (i) a variable light chain sequence having at least 85%, at least 90%, at least 95%, or at least 98% sequence identity to the amino acid sequence of SEQ ID NO: 64; (ii) a variable heavy chain sequence having at least 85%, at least 90%, at least 95%, or at least 98% sequence identity to the amino acid sequence of SEQ ID NO: 67; or (iii) a variable light chain sequence as in (i) and a variable heavy chain sequence as in (ii); or
e. (i) a variable light chain sequence having at least 85%, at least 90%, at least 95%, or at least 98% sequence identity to the amino acid sequence of SEQ ID NO: 65; (ii) a variable heavy chain sequence having at least 85%, at least 90%, at least 95%, or at least 98% sequence identity to the amino acid sequence of SEQ ID NO: 68; or (iii) a variable light chain sequence as in (i) and a variable heavy chain sequence as in (ii); or
f. (i) a variable light chain sequence having at least 85%, at least 90%, at least 95%, or at least 98% sequence identity to the amino acid sequence of SEQ ID NO: 66; (ii) a variable heavy chain sequence having at least 85%, at least 90%, at least 95%, or at least 98% sequence identity to the amino acid sequence of SEQ ID NO: 69; or (iii) a variable light chain sequence as in (i) and a variable heavy chain sequence as in (ii).

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, (β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.” In some embodiments, the anti-IL31 antibody is purified using chromatography, such as size exclusion chromatography, ion exchange chromatography, protein A column chromatography, hydrophobic interaction chromatography, and CHT chromatography.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

The term “IL31 signaling function” refers to any one of or combination of the downstream activities that occurs when IL31 binds its receptor or receptor complex. In some embodiments, the IL31 signaling function comprises activation of Janus kinase (Jak) 1 or Jak 2 signaling molecules. In some embodiments, the IL31 signaling function comprises phosphorylation of STAT-3 or STAT-5 proteins. In some embodiments, the IL31 signaling function comprises activating the ERK1/2 MAP kinase signaling pathway. In some embodiments, the IL31 signaling function comprises activating the PI3K/AKT signaling pathway. In some embodiments, the IL31 signaling function comprises activating the Jak1/2 signaling pathway.

“STAT phosphorylation” means the post-expression modification of a STAT protein by phosphorylation. For example, “STAT-3 phosphorylation” refers to the phosphorylation of STAT-3 and “STAT-5 phosphorylation” refers to the phosphorylation of STAT-5. In some embodiments, the phosphorylation of STAT-3 is measured by immune-blot analysis. For example, cells (e.g., canine monocytic DH82 cells) are plated into a 96-well cell culture plate at a density of 1×10⁵ cells per well in growth media (e.g., MEM, Life Technologies®) containing 15% heat-inactivated fetal bovine serum, 2 mmol/L GlutaMax, 1 mmol/L sodium pyruvate, and 10 nm/mL canine interferon-c (R&D Systems, Minneapolis, Minn., USA) for 24 hours at 37° C. in the presence of anti-IL31 antibody as described herein. Immuno-blot analysis of the cell lysate using anti-phospho STAT-3 and anti-STAT-3 antibodies (R&D Systems) were used to detect the concentration of phosphorylated STAT-3 and unphosphorylated STAT-3 relative to each other and compared to a beta-actin control. Methods for determining the concentration of proteins, either qualitatively or quantitatively, by immunoblot are understood by persons of skill in the art. In some embodiments, relative concentration is determined by qualitatively by visual inspection of the immunoblot. In some embodiments, the concentration of phosphorylated STAT-3 and unphosphorylated STAT-3 is quantitatively determined by digitally imaging an immunoblot, determining the intensity of the bands, and using a linear standard curve of known concentrations of STAT-3 protein to back calculate the concentration of phosphorylated or unphosporylated STAT-3 in a sample.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value. In some embodiments, an IL31 antibody may reduce IL31 signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to IL31 signaling function in the absence of the antibody, as measured by a reduction in STAT-3 phosphorylation. In some embodiments, the reduction in IL31 signaling function or the reduction in STAT-3 phosphorylation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Anti-bacterial agents (e.g, bacteriostatic reagents, such benzyl alcohol, may be included. Thus, the invention provides pharmaceutical compositions in solid or liquid form. The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

In some embodiments, the pharmaceutically acceptable carrier or the pharmaceutical composition has a pH of from 5.0 to 6.2, from 5.0 to 6.0, or from 5.3 to 5.7. In some embodiments, the pharmaceutically acceptable carrier or the pharmaceutical composition has a pH of 5.0, 5.1, 5.2, 5.3, 5.4, 5.5, 5.6, 5.7, 5.8, 5.9, 6.0, 6.1, or 6.2

In some embodiments, the pharmaceutically acceptable carrier or the pharmaceutical composition comprises L-histidine, sodium chloride, and polysorbate 80.

In some embodiments, the pharmaceutically acceptable carrier or the pharmaceutical composition comprises sodium chloride at a concentration of from 80 nM to 200 nM, of from 100 nM to 180 nM, of from 100 nM to 175 nM, of from 110 nM to 170 nM, of from 120 nM to 160 nM, of from 120 nM to 150 nM, of from 130 nM to 150 nM, of from 130 nM to 160 nM, of 100 nM, of 80 nM, of 110 nM, of 120 nM, of 130 nM, of 140 nM, of 150 nM, of 160 nM, of 170 nM, of 180 nM, or of 200 nM.

In some embodiments, the pharmaceutically acceptable carrier or the pharmaceutical composition comprises polysorbate 80 at a concentration of from 0.005 mg/mL to 0.5 mg/mL, of from 0.01 mg/mL to 0.1 mg/mL, of from 0.1 mg/mL to 0.5 mg/mL, of from 0.005 mg/mL to 0.01 mg/mL, of 0.1 mg/mL, of 0.2 mg/mL, of 0.3 mg/mL, of 0.4 mg/mL, of 0.05 mg/mL, of 0.06 mg/mL, of 0.07 mg/mL, of 0.08 mg/mL, of 0.09 mg/mL, or of 0.1 mg/mL.

In some embodiments, the pharmaceutically acceptable carrier or the pharmaceutical composition comprises L-histidine at a concentration of from 5 mM to 100 mM, of from 10 mM to 50 mM, of from 20 mM to 30 mM, of from 10 mM to 30 mM, of from 20 mM to 80 mM, of from 30 mM to 70 mM, of from 40 mM to 60 mM, of 10 mM, of 15 mM, of 20 mM, of 25 mM, of 30 mM, of 40 mM, or of 50 mM.

In some embodiments, the pharmaceutically acceptable carrier or the pharmaceutical composition comprises m-cresol or benzyl alcohol. In some embodiments, the concentration of m-cresol is about 0.2%, of from about 0.1% to about 0.3%, of from about 0.08% to about 0.25%, or of from about 0.05% to about 0.25%. In some embodiments, the concentration of benzyl alcohol is about 1%, of from about 0.5% to about 2%, of from about 0.2% to about 2.5%, of about 1% to about 5%, of about 0.5% to about 5%, or of about 1% to about 3%.

In some embodiments, the pharmaceutically acceptable carrier or the pharmaceutical composition comprises a sugar. In some embodiments, the sugar is sucrose, trehalose, D-mannitol, maltose, and/or sorbitol. In some embodiments, the pharmaceutically acceptable carrier or the pharmaceutical composition comprises a sugar at a concentration of 0.5% to 20%, of from 1% to 10%, of from 1% to 5%, of from 1% to 3%, of 0.5%, of 1%, of 2%, of 3%, of 4%, of 5%, or of 10%.

In some embodiments, the pharmaceutically acceptable carrier or a pharmaceutical composition comprises an anti-bacterial agent. In some embodiments, the pharmaceutically acceptable carrier or pharmaceutical composition comprises m-cresol or methylparaben. In some embodiments, the pharmaceutically acceptable carrier or pharmaceutical composition comprises 0.2% m-cresol. In some embodiments, the pharmaceutically acceptable carrier or pharmaceutical composition comprises 0.9% methylparaben.

Uses of Antibodies and Pharmaceutical Compositions

The antibodies or pharmaceutical compositions comprising the antibodies of the invention may be useful for treating an IL-31-induced condition. As used herein, an “IL31-induced condition” means a disease associated with, caused by, or characterized by, elevated levels or altered gradients of IL31 concentration. Such IL31-induced conditions include, but are not limited to, a pruritic or an allergic disease. In some embodiments, the IL31-induced condition is atopic dermatitis, pruritus, asthma, psoriasis, scleroderma, or eczema. An IL31-induced condition may be exhibited in a companion animal, including, but not limited to, canine, feline, or equine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an anti-IL31 antibody or pharmaceutical compositions comprising it can be utilized in accordance with the methods herein to treat IL31-induced conditions. In some embodiments, an anti-IL31 antibody or pharmaceutical compositions is administered to a companion animal, such as a canine, a feline, or equine, to treat an IL31-induced condition.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an anti-IL31 antibody or pharmaceutical composition comprising an anti-IL31 antibody is administered parenterally, by subcutaneous administration, intravenous infusion, or intramuscular injection. In some embodiments, an anti-IL31 antibody or pharmaceutical composition comprising an anti-IL31 antibody is administered as a bolus injection or by continuous infusion over a period of time. In some embodiments, an anti-IL31 antibody or pharmaceutical composition comprising an anti-IL31 antibody is administered by an intramuscular, an intraperitoneal, an intracerebrospinal, a subcutaneous, an intra-arterial, an intrasynovial, an intrathecal, or an inhalation route.

Anti-IL31 antibodies described herein may be administered in an amount in the range of 0.1 mg/kg body weight to 100 mg/kg body weight per dose. In some embodiments, anti-IL31 antibodies may be administered in an amount in the range of 0.5 mg/kg body weight to 50 mg/kg body weight per dose. In some embodiments, anti-IL31 antibodies may be administered in an amount in the range of 1 mg/kg body weight to 10 mg/kg body weight per dose. In some embodiments, anti-IL31 antibodies may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, or in the range of 5 mg/kg body weight to 50 mg/kg body weight.

An anti-IL31 antibody or a pharmaceutical composition comprising an anti-IL31 antibody can be administered to a companion animal at one time or over a series of treatments. For example, an anti-IL31 antibody or a pharmaceutical composition comprising an anti-IL31 antibody may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment. Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with an anti-IL31 antibody or a pharmaceutical composition comprising an anti-IL31 antibody, a Jak inhibitor, a PI3K inhibitor, an AKT inhibitor, or a MAPK inhibitor. In some embodiments, the method comprises administering in combination with an anti-IL31 antibody or a pharmaceutical composition comprising an anti-IL31 antibody, an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11a antibody, anti-IL6R antibody, anti-α4-Intergrin antibody, an anti-IL12 antibody, an anti-IL10 antibody, or an anti-BlyS antibody.

Provided herein are methods of exposing to a cell an anti-IL31 antibody or a pharmaceutical composition comprising an anti-IL31 antibody under conditions permissive for binding of the antibody to IL31. In some embodiments, the cell is exposed to the antibody or pharmaceutical composition ex vivo. In some embodiments, the cell is exposed to the antibody or pharmaceutical composition in vivo. In some embodiments, a cell is exposed to the anti-IL31 antibody or the pharmaceutical composition under conditions permissive for binding of the antibody to intracellular IL31. In some embodiments, a cell is exposed to the anti-IL31 antibody or the pharmaceutical composition under conditions permissive for binding of the antibody to extracellular IL31. In some embodiments, a cell may be exposed in vivo to the anti-IL31 antibody or the pharmaceutical composition by any one or more of the administration methods described herein, including but not limited to, intraperitoneal, intramuscular, intravenous injection into the subject. In some embodiments, a cell may be exposed ex vivo to the anti-IL31 antibody or the pharmaceutical composition by exposing the cell to a culture medium comprising the antibody or the pharmaceutical composition. In some embodiments, the permeability of the cell membrane may be affected by the use of any number of methods understood by those of skill in the art (such as electroporating the cells or exposing the cells to a solution containing calcium chloride) before exposing the cell to a culture medium comprising the antibody or the pharmaceutical composition.

In some embodiments, the binding results in a reduction of IL31 signaling function by the cell. In some embodiments, an IL31 antibody may reduce IL31 signaling function in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to IL31 signaling function in the absence of the antibody, as measured by a reduction in STAT-3 phosphorylation. In some embodiments, the reduction in IL31 signaling function or the reduction in STAT-3 phosphorylation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the anti-IL31 antibodies, polypeptides and polynucleotides for detection, diagnosis and monitoring of an IL31-induced condition. Provided herein are methods of determining whether a companion animal will respond to anti-IL31 antibody therapy. In some embodiments, the method comprises detecting whether the animal has cells that express IL31 using an anti-IL31 antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with an anti-IL31 antibody and the binding between the antibody and the cell is determined. When the test cells show binding activity as compared to a reference cell of the same tissue type, it may indicate that the subject would benefit from treatment with an anti-IL31 antibody. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or p-glactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the anti-IL31 antibodies need not be labeled, and the presence thereof can be detected using a second labeled antibody which binds to the first anti-IL31 antibody. In some embodiments, the anti-IL31 antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The anti-IL31 antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography. The antibody may also be used as staining reagent in pathology using techniques well known in the art. In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

The following examples illustrate particular aspects of the disclosure and are not intended in any way to limit the disclosure.

### EXAMPLES

### Example 1: Identification of Mouse Monoclonal Antibodies that Bind to Canine IL31

Canine IL31 gene encoding IL31 protein (SEQ ID NO: 22) was synthesized with poly-His tag on the C-terminal and cloned into a mammalian expression vector. The plasmid that carries canine IL31 gene was transfected to 293 cells.

The supernatant containing canine IL31 protein was collected and filtered. Canine IL31 was affinity purified using Ni-NTA column (CaptivA® Protein A Affinity Resin, Repligen).

Mouse monoclonal antibodies were identified using standard immunization using canine IL31 produced by 293 cells as immunogen. Two different adjuvants were used during immunizations (Antibody Solutions, Sunnyvale, Calif.) and monoclonal antibodies were obtained through standard hybridoma technology. Enzyme linked immunosorbent assay (ELISA) was developed to screen the clones that produce IL31 binding antibodies. First canine IL31 was biotinylated and then it was introduced to streptavidin-coated wells. Immunized serum was then added to the wells followed by washing and detection with HRP-conjugated anti-mouse antibodies. The presence of canine IL31 binding antibody developed a positive signal. Of the thousands of clones tested by ELISA, 170 clones having the highest binding affinity based on signal intensity were selected for further testing by biosensor assay (Forte Bio Octet). Biotinylated canine IL31 was bound to the sensor tip and hybridoma clone supernatants containing anti-canine IL31 antibodies were selected based on a slow off-rate (the rate of dissociation between antibody and ligand). The binding affinity of the top 19 candidates were measured at single concentration and reported as the equilibrium dissociation constant (Kd) after the antibody concentrations were measured by protein A titer assay using Biosensor Octet. The Kds of the top 19 candidates were each less than 10 nM.

Furthermore, each of the 170 clones having high binding activity based on ELISA was tested for neutralization activity. The cell-based functional assay described below in Example 4, was performed to assess activity of the top candidates in reducing canine IL31-mediated pSTAT signaling using canine DH82 cells. Four top clones (M14, M18, M19, and M87) were selected for further investigation. Notably, the majority of the high affinity binders identified by ELISA were not functional.

### Example 2: Identification of DNA Sequences Encoding VH and VL of Monoclonal Antibodies

Hybridoma cells producing M14, M18, M19 and M87 were pelleted. RNA was extracted and oligonucleotide primers for amplifying mouse immunoglobulin (Ig) variable domains were used to obtain cDNA using standard techniques. The variable heavy chain (VH) and variable light chain (VL) of each of the four clones were sequenced and analyzed by sequence alignment (FIG. 1; SEQ ID NOs 36 to 43). Notably, three of the four active antibodies (M14, M18, and M19) share the same six CDR sequences, with the exception that CDR-L1 of M18 has an isoleucine at position 14 (SEQ ID NO: 63), where M14 and M19 have a methionine at position 14 (SEQ ID NO: 8), and CDR-H2 of M18 has a tyrosine at position 9 (SEQ ID NO: 62 and SEQ ID NO: 87), where M14 and M19 have a asparatic acid at position 14 (SEQ ID NO: 2 and SEQ ID NO: 89). The similarity in CDR sequences suggests that M14, M18, and M19 share a common epitope.

### Example 3: Expression and Purification of Murine-Canine Chimeric and Caninized IL31-mAb M14 from CHO Cells

DNA sequences encoding a chimeric antibody were designed for a fusion of murine M14 VH (SEQ ID NO: 25) and murine VL (SEQ ID NO: 24) to canine constant heavy chain and canine constant light chain. The nucleotide sequences were synthesized chemically and inserted into an expression vector suitable for transfection into a CHO host cell. After transfection into CHO cells, the light chain or heavy chain protein or both were secreted from the cell. For example, chimeric M14 that uses canine IgG-B was purified by single step Protein A column chromatography.

Murine M14 VH and VL were caninized by searching and selecting proper canine germline antibody sequences as a template for CDR grafting, followed by protein modeling. Caninized M14 IgG-B (SEQ ID NO: 18 and SEQ ID NO: 21) was readily expressed and purified in a single step with a protein A column or other chromatographic methods, such as ion exchange column chromatography, hydrophobic interaction column chromatography, mixed mode column chromatography such as CHT, or multimodal mode column chromatography such as CaptoMMC. Low pH or other viral inactivation and viral removal steps can be applied. The purified protein is admixed with excipients, and sterilized by filtration to prepare a pharmaceutical composition of the invention. The pharmaceutical composition is administered to a dog with an atopic dermatitis in an amount sufficient to bind to inhibit IL31.

The vectors were then used to perform pilot-scale transfection in CHO-S cells using the FreestyleMax™ transfection reagent (Life Technologies). The supernatant was harvested by clarifying the conditioned media. Protein was purified with a single pass Protein A chromatography step and used for further investigation.

### Example 4: Demonstration of IL31 Binding Activity

This example demonstrates that antibodies of the invention, illustrated with the chimeric M14 (SEQ ID NO:26 and SEQ ID NO:27) and caninized M14 (SEQ ID NO: 18 and SEQ ID NO:21) bind canine IL31 with kinetics requisite for therapeutic activity.

The binding analysis was performed using a biosensor Octet as follows. Briefly, canine IL31 was biotinylated at primary amine groups using EZ-Link™ NHS-LC-Biotin (Thermo Scientific, Catalog No. 21336) or at glycan groups using EZ-Link™ Biotin-LC-Hydrazide (ThermoFisher Scientific, Catalog No. 21340) according to the manufacturer's instructions. The free unreacted biotin was removed from biotinylated IL31 by extensive dialysis. Biotinylated canine IL31 was captured on streptavidin sensor tips. The association of four different concentrations (400, 200, 66.6, and 33 nM) of chimeric and caninized M14 antibody and human and canine IL31 (amine-conjugated-biotin) and the association of 100 nM of caninized M14 antibody and canine IL31 (glycan-conjugated-biotin) was monitored for ninety seconds. Dissociation was monitored for 600 seconds. A buffer only blank curve was subtracted to correct for any drift. The data were fit to a 2:1 binding model using ForteBio™ data analysis software to determine the kₒₙ, k_{off}, and the Kd. The buffer for dilutions and all binding steps was: 20 mM phosphate, 150 mM NaCl, pH 7.2.

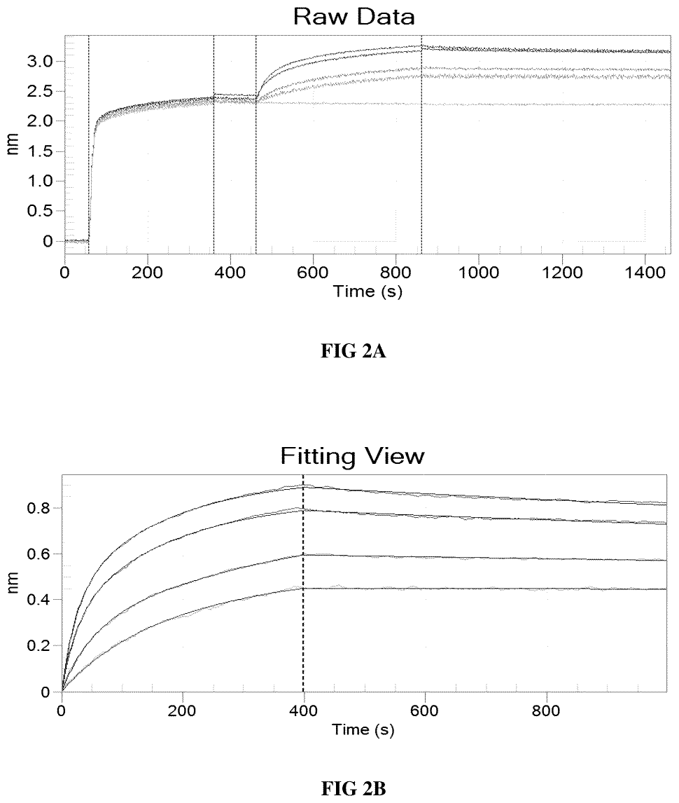

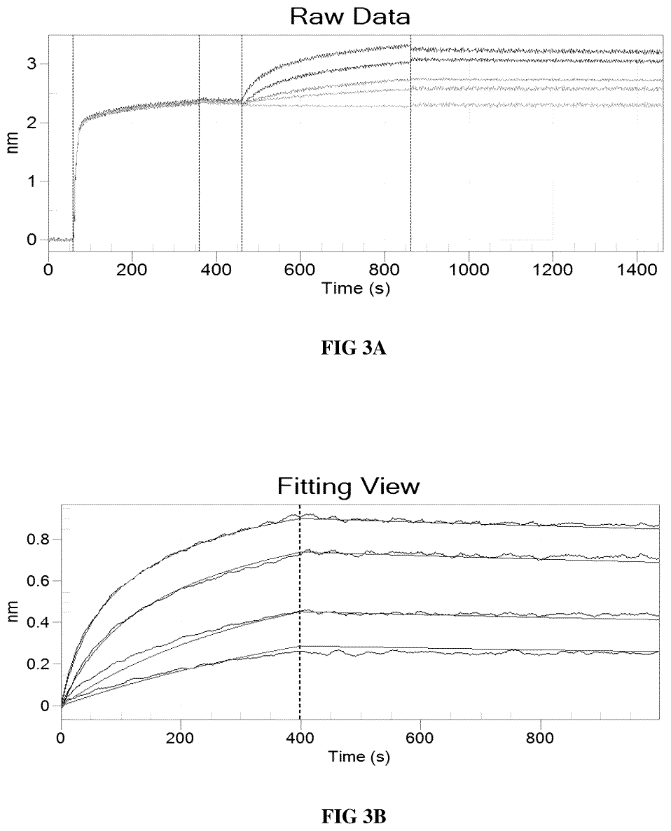

Canine IL31 with C-terminal polyHis tag was expressed and purified from CHO-S cells. Human IL31 was obtained from Sino Biologicaland Streptavidin biosensors was obtained from ForteBio (Cat. #18-509). The binding kinetics were as follows: For the ligand canine IL31 (amine-conjugated-biotin), the Kd (M) for chimeric M14 was <1.0×10⁻¹¹ (FIG. 2) and <1.0×10⁻¹¹ (FIG. 3) for caninized M14. For the ligand canine IL31 (glycan-conjugated-biotin), the Kd (M) for caninized M14 was <1.0×10⁻¹² and k_{off} (1/s) was <1.0×10⁻⁷.

Chimeric M14 and caninized M14 had no obvious binding signal with human IL31. Thus, the Kd could not be measured.

### Example 5: Demonstration that M14 Inhibits Canine IL31 Signaling

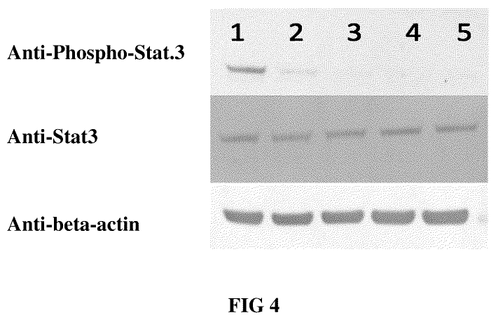

After binding to its IL31 receptor, IL-31 activates Janus kinase (Jak) 1 and Jak2 signaling molecules. In turn, activated Jaks stimulate the phosphorylation of downstream signaling STAT-3 and STAT-5. Anti-phospho-Stat3 immuno-blot analysis was used to detect anti-IL31 activity from a protein A-purified fraction of cell-free culture medium (Gonzales et. al. Vet Dermatol 2013; 24: 48-e12). In Brief, the canine monocytic DH82 cells (American Type Culture Collection, Manassas, Va., USA) were plated into 96-well flat-bottomed cell culture plates at a density of 1×10⁻⁵ cells per well in MEM growth media (Life Technologies) containing 15% heat-inactivated fetal bovine serum, 2 mmol/L GlutaMax, 1 mmol/L sodium pyruvate, and 10 ng/mL canine interferon-c (R&D Systems, Minneapolis, Minn., USA) for 24 h at 37° C. In this experiment, concentration of canine IL31-Fc was 5 ng/mL (8 nM). Anti-phospho STAT-3 and anti-STAT-3 antibodies were purchased from R&D Systems. Anti-beta actin antibody was from Sigma-Aldrich. As shown in FIG. 4, canine IL31 signaling decreased (as evidenced by a reduction in STAT-3 phosphorylation) as the concentration of caninized M14 exposed to the cells increased (lane 1: no anti-IL31 antibody; Lane 2: 3.3 nM; Lane 3: 6.6 nM; Lane 4: 9.9 nM; and Lane 5: 13.2 nM).

### Example 6: Identification of M14 Canine IL31 Binding Epitope

To identify the canine IL31 epitope recognized by M14, multiple GST canine IL31 fragment fusion molecules were generated and proteins were expressed intracellularly in E. coli. After the GST fusion proteins were transferred to a membrane, chimeric M14 was used to probe the membrane. A positive signal resulted when the IL31 fragment contained the epitope.

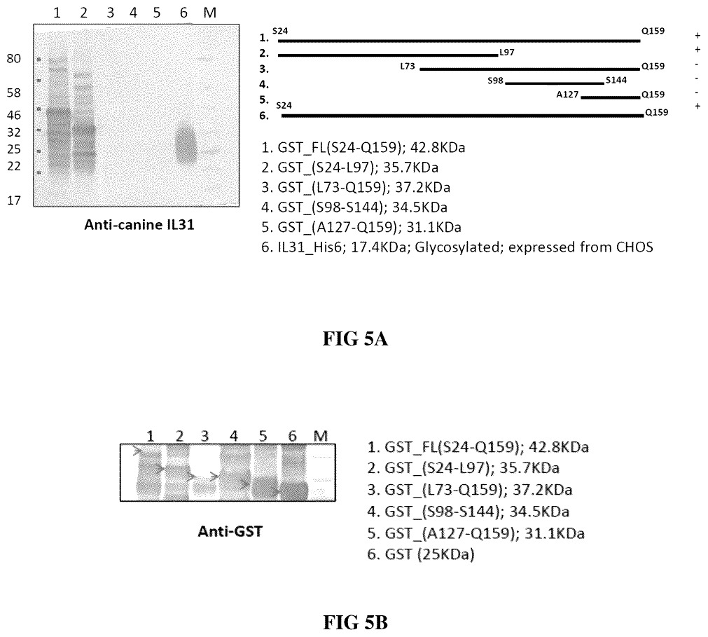

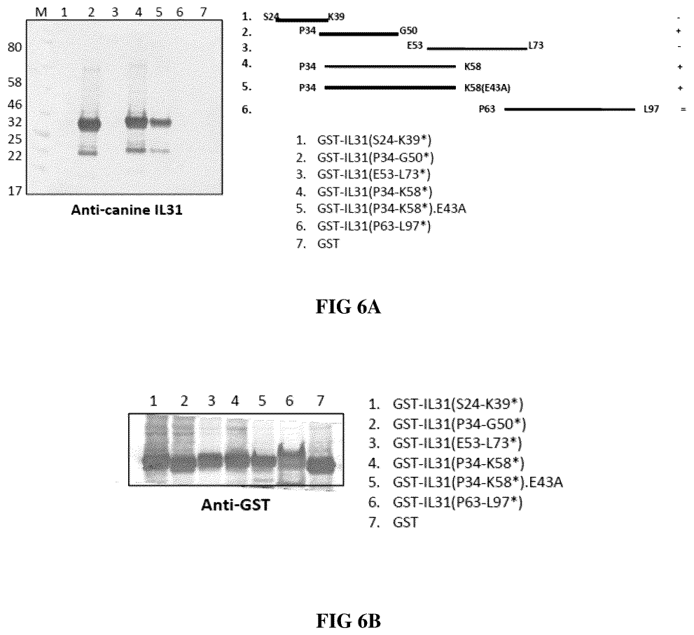

FIG. 5 combined with FIG. 6 demonstrated M14 can recognize the minimal fragment (SEQ ID NO: 23).

### Example 7: Demonstrating M14 Cross Reacts to Feline IL31

To examine whether M14 antibody recognizes feline IL31 (SEQ ID NO: 28) or equine IL31 (SEQ ID NO: 29), each protein was fused to human Fc and expressed in mammalian 293 cells. The partially purified proteins were blotted to membrane and probed with M14 antibody. The immunoblot of FIG. 1 demonstrates that M14 binds to feline IL31. The immunoblot assay did not detect binding between M14 and equine IL31. However, biolayer interferometry analysis revealed that M14 antibody binds equine IL31, but with a lesser affinity. The preliminary Kd measurement using biotinylated equine IL31 immobilized to the sensor revealed that the affinity (Kd) is approximately 10 to 50 nM.

### Example 8: Felinized M14

M14 variable light chain was felinized as (SEQ ID NO: 32) and M14 variable heavy chain was felinized as (SEQ ID NO: 33). First, the mouse heavy chain variable and light chain variable sequences were used to search proper variants of feline VH and VL. The proper feline frames were chosen to graft CDRs. They are further optimized using structural modeling. The felinized VH and VL were fused to a feline IgG heavy chain constant domains (CH1, CH2, and CH3) and feline light chain constant domain (CL1).

Feline M14 chimeric antibody (SEQ ID NO: 30 and SEQ ID NO: 31) or felinized M14 antibody (SEQ ID NO: 34 and SEQ ID NO: 35) can be as administered to cats for treatment of an IL31-induced condition.

### Example 9: Identification of M14 Canine IL31 Binding Epitope

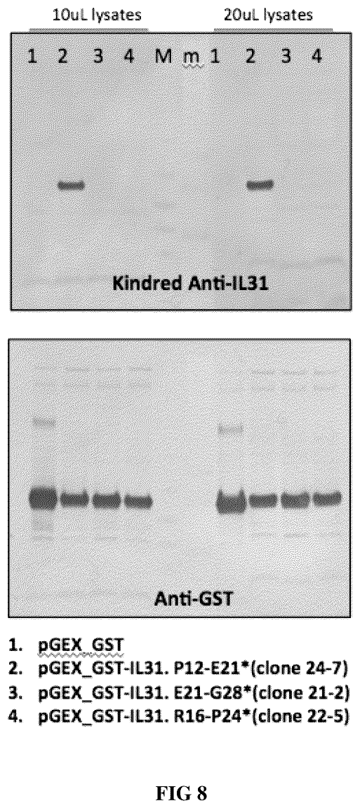

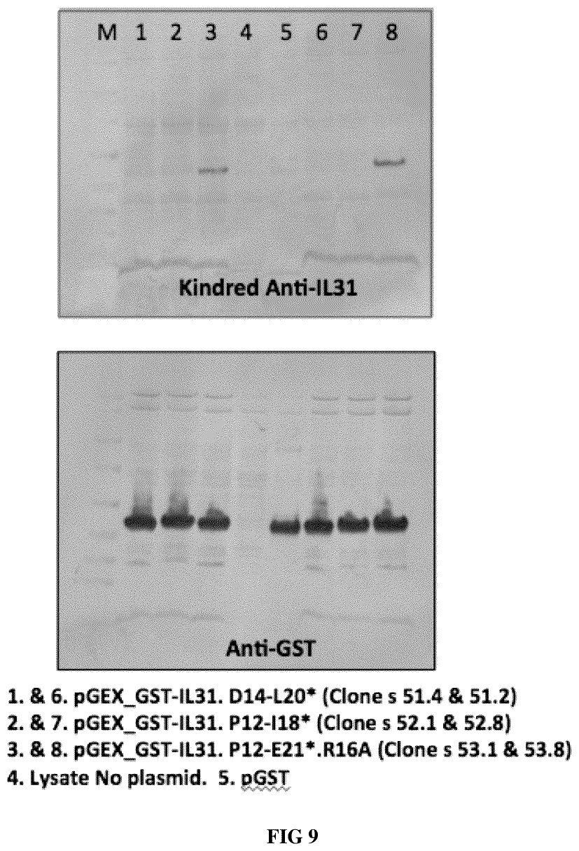

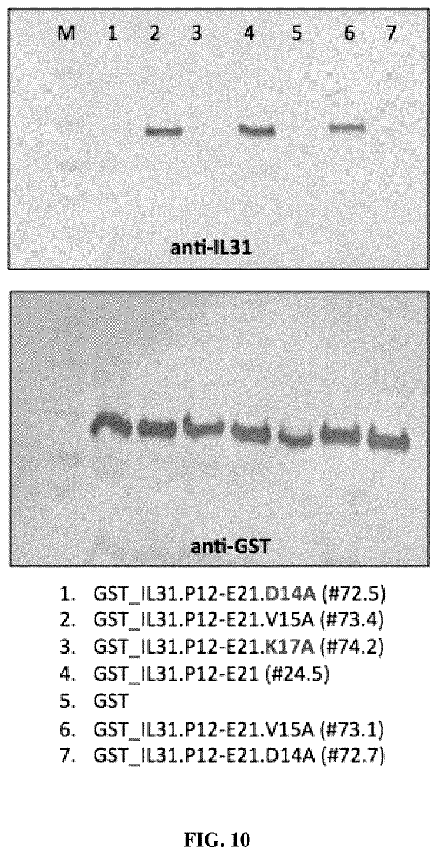

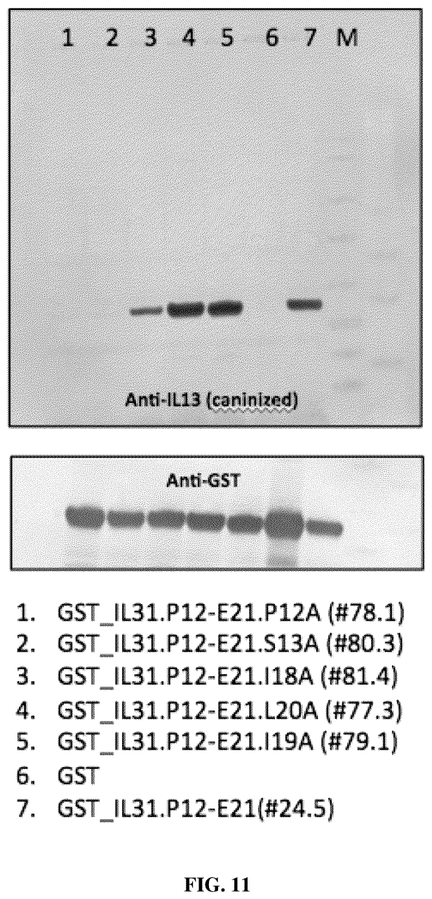

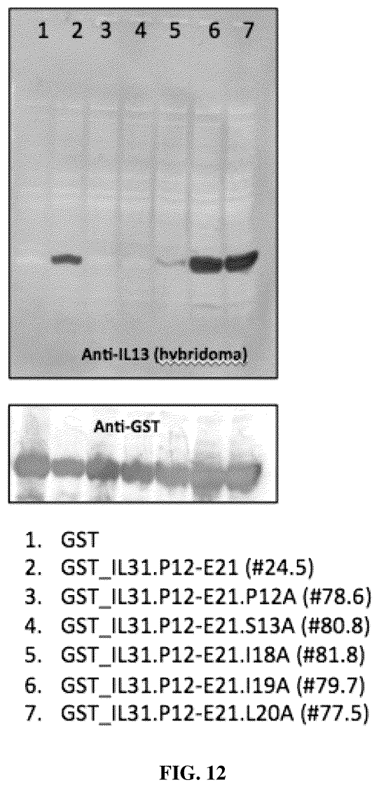

To further identify amino acid residues of the canine IL31 epitope recognized by M14, multiple GST canine IL31 epitope fragment fusion molecules carrying alanine mutations were expressed in E. coli. FIGS. 8-12 show immunoblots of fine epitope mapping of canine IL31-GST fusion proteins probed with anti-canine IL31-mAb (M14) or caninized M14 (top panels) and anti-GST antibody control (bottom panels). The epitope fragment tested in each lane is listed below the immunoblots. The fragment name indicates the range of amino acids of mature canine IL31 (SEQ ID NO: 44) tested and the position of the alanine mutation, if one was included. A positive signal resulted when the IL31 fragment contained the wildtype epitope, while a negative signal resulted when the IL31 fragment contained an alanine substitution at a residue important for antibody-ligand interaction.

The results of the epitope mapping study are summarized in Table 3, below. The results suggest that P12, S 13, D14, and K17 of mature canine IL31 (SEQ ID NO: 44) are involved in binding of antibody M14 and that R16 and 118 are partially involved in M14 recognition. In sum, the results of this study suggest that the motif of an IL31 polypeptide that bind the CDRs of antibody M14 is: PSDX₁×₂KI, where X₁ and X₂ are variable (SEQ ID NO: 45).

TABLE 3

| Amino Acid | Does substitution |
|---|---|
| Substitution | prevent binding? |
| P12A | Yes |
| S13A | Yes |
| D14A | Yes |
| V15A | No |
| R16A | Partially |
| K17A | Yes |
| I18A | Partially |
| I19A | No |
| L20A | No |

### Example 10: M14 Specifically Binds to IL31 of Other Species Having the PSDX1×2KI Epitope Motif

The IL31 epitope motif recognized by M14 (PSDXiX₂KI; SEQ ID NO: 45) is not present in the IL31 amino acid sequences of human (SEQ ID NO: 46) or mouse (SEQ ID NO: 61). However, the motif was identified in several other species, including feline:
Felis catus (XP_011286140.1; SEQ ID NO: 28)
Odobenus rosmarus divergens (XP_004395998.1; SEQ ID NO: 47)
Papio Anubis (XP_003907358.1; SEQ ID NO: 49)
Ursus maritimus (XP_008687166.1; SEQ ID NO: 51)
Leptonychotes weddelihi (XP_006746595.1; SEQ ID NO: 52)
Panthera tigris altaica (XP_007079636.1; SEQ ID NO: 53)
Acinonyx jubatus (XP_014919275.1; SEQ ID NO: 54)
Macaca fascicularis (EHH66805.1; SEQ ID NO: 55)
Macaca mulatta (EHH21279.1; SEQ ID NO: 56)
Mandrillus leucophaeus (XP_011819882.1; SEQ ID NO: 57)
Chlorocebus sabaeus (XP_008003211.1; SEQ ID NO: 58)
Cercocebus atys (XP_011926625.1; SEQ ID NO: 59)
Rhinopithecus roxellana (XP_010366647.1; SEQ ID NO: 60)

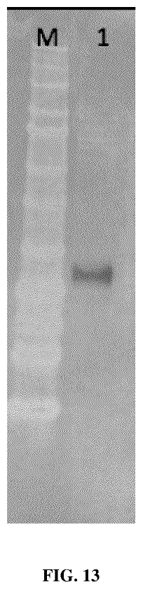

Walrus IL31 (SEQ ID NO: 47) and oliver baboon IL31 (SEQ ID NO: 49) possess the PSDX₁×₂KI epitope (SEQ ID NO: 45) that can be recognized by antibody M14. To facilitate protein purification, C-terminal His tag was added to walrus IL31 (SEQ ID NO: 48) and to oliver baboon IL31 (SEQ ID NO: 50). Western blot analysis confirmed that M14 binds walrus IL31 (FIG. 13). M14 antibody or its derivatives can be used for therapeutic and diagnostic agents for an IL31-induced disease in any of the above listed species.

### Example 11: Thermostability of Caninized M14 Antibody

Thermostability of caninized M14 antibody was compared to Zoetis' CYTOPOINT™, a commercially available anti-IL31 antibody, across a broad range of pH was analyzed using differential scanning fluorescence (DSF). The melting temperature (Tm) of each antibody at the different pHs is listed in Table 4, below. Both CYTOPOINT™ and caninized M14 antibodies were buffer exchanged into the assay buffer listed in Table 4 using a PD Minitrap™ G-25 column (GE Healthcare). Tm of each antibody was evaluated using the same buffer and protein concentration. Caninized M14 antibody exhibited improved thermostability compared to CYTOPOINT™ across a broad pH range. Furthermore, under stress conditions at 55° C. for 2 days at 0.22 mg/ml antibody, CYTOPOINT™ precipitated, while no precipitates were observed with caninized M14 antibody.

TABLE 4

| | | Caninized M14 | CYTOPOINT ™ |
|---|---|---|---|
| | | Melting | Melting |
| pH | | temperature | temperature |
| tested | Assay Buffer | (Tm° C.) | (Tm° C.) |
| 3 | 0.1M NaAc | 55.44 | 49.21 |
| 4 | 0.1M NaAc | 61.68 | 57.54 |
| 5 | 0.1M NaAc | 66.84 | 61.71 |
| 6 | 0.1M NaPO₄ | 68.20 | 62.84 |
| 7 | 0.1M NaPO₄ | 67.25 | 61.76 |
| 7.2 | 2xPBS | 66.71 | 60.98 |
| 8 | 0.1M NaPO₄ | 65.08 | 60.08 |
| 9 | 0.1M TrisHCl | 64.25 | 59.25 |

### Example 12. Caninized M14 Antibody Buffer Formulations

Thermostability of caninized M14 antibody in various buffer formulations was analyzed. Buffers containing sodium phosphate, sodium acetate, or L-histidine were considered. Other formulation variables included different pHs (pH 5.2, 5.5, 6.0, 6.5, and 7.0), different concentrations of sodium chloride (50 mM and 140 mM), different polysorbates (polysorbate 20 and polysorbate 80), and different anti-bacterial agents (m-cresol and methylparaben). The melting temperature (Tm) of caninized M14 antibody in each buffer was measured by differential scanning fluorescence (DSF) technique from 20° C. to 95° C. Table 5 lists Tm values of caninized M14 antibody in the various buffers tested.

TABLE 5

| | | Melting |
|---|---|---|
| Formulation | | temperature |
| Designation | Buffer Formulation | (Tm° C.) |
| A1 | 20 mM sodium phosphate | 67.6 |
| | 140 mM sodium chloride | |
| | Polysorbate 80 (0.05 mg/mL) | |
| | pH 6.0 | |
| A2 | A1 + m-cresol (0.2%) | 65.0 |
| A3 | A1 + Methyparaben 0.9 mg/mL | 67.3 |
| A4 | 20 mM sodium phosphate | 66.9 |
| | 140 mM sodium chloride | |
| | Polysorbate 20 (0.05 mg/mL) | |
| | pH 6.0 | |
| A5 | A4 + 0.2% m-cresol | 64.4 |
| A6 | A4 + Methyparaben 0.9 mg/mL | 67.3 |
| A7 | 20 mM sodium phosphate | 66.3 |
| | 50 mM sodium chloride | |
| | Polysorbate 80 (0.05 mg/mL) | |
| | pH 6.0 | |
| A8 | A7 + 0.2% m-cresol | no peak* |
| A9 | A7 + Methyparaben 0.9 mg/mL | 66.1 |
| A10 | 20 mM sodium phosphate | 65.8 |
| | 50 mM sodium chloride | |
| | Polysorbate 20 (0.05 mg/mL) | |
| | pH 6.0 | |
| A11 | A10 + 0.2% m-cresol | 63.6 |
| A12 | A10 + Methyparaben 0.9 mg/mL | 65.5 |
| B1 | 20 mM sodium phosphate | 68.0 |
| | 140 mM sodium chloride | |
| | Polysorbate 80 (0.05 mg/mL) | |
| | pH 7.0 | |
| B2 | B1 + m-cresol (0.2%) | 66.2 |
| B3 | B1 + Methyparaben 0.9 mg/mL | 68.1 |
| B4 | 20 mM sodium phosphate | 68.1 |
| | 140 mM sodium chloride | |
| | Polysorbate 20 (0.05 mg/mL) | |
| | pH 7.0 | |
| B5 | B4 + 0.2% m-cresol | 64.6 |
| B6 | B4 + Methyparaben 0.9 mg/mL | 67.2 |
| B7 | 20 mM sodium phosphate | 67.6 |
| | 50 mM sodium chloride | |
| | Polysorbate 80 (0.05 mg/mL) | |
| | pH 7.0 | |
| B8 | A7 + 0.2% m-cresol | 64.7 |
| B9 | B7 + Methyparaben 0.9 mg/mL | 66.9 |
| B10 | 20 mM sodium phosphate | 67.3 |
| | 50 mM sodium chloride | |
| | Polysorbate 80 (0.05 mg/mL) | |
| | pH 7.0 | |
| B11 | B10 + 0.2% m-cresol | 64.8 |
| B12 | B10 + Methyparaben 0.9 mg/mL | 67.4 |
| C1 | 20 mM sodium acetate | 67.9 |
| | 140 mM sodium chloride | |
| | Polysorbate 80 (0.05 mg/mL) | |
| | pH 5.2 | |
| C2 | C1 + m-cresol (0.2%) | 65.4 |
| C3 | C1 + Methyparaben 0.9 mg/mL | 67.6 |
| C4 | 20 mM sodium acetate | 67.7 |
| | 140 mM sodium chloride | |
| | Polysorbate 20 (0.05 mg/mL) | |
| | pH 5.2 | |
| C5 | C4 + 0.2% m-cresol | 64.7 |
| C6 | C4 + Methyparaben 0.9 mg/mL | 67.7 |
| C7 | 20 mM sodium acetate | 66.6 |
| | 50 mM sodium chloride | |
| | Polysorbate 80 (0.05 mg/mL) | |
| | pH 5.2 | |
| C8 | C7 + 0.2% m-cresol | no peak* |
| C9 | C7 + Methyparaben 0.9 mg/mL | 66.9 |
| C10 | 20 mM sodium acetate | 68.0 |
| | 50 mM sodium chloride | |
| | Polysorbate 20 (0.05 mg/mL) | |
| | pH 5.2 | |
| C11 | C10 + 0.2% m-cresol | 64.7 |
| C12 | C10 + Methyparaben 0.9 mg/mL | 67.7 |
| D1 | 20 mM L-Histidine | 69.5 |
| | 140 mM sodium chloride | |
| | Polysorbate 80 (0.05 mg/mL) | |
| | pH 5.5 | |
| D2 | D1 + m-cresol (0.2%) | 68.1 |
| D3 | D1 + Methyparaben 0.9 mg/mL | 69.6 |
| D4 | 20 mM L-Histidine | 69.6 |
| | 140 mM sodium chloride | |
| | Polysorbate 20 (0.05 mg/mL) | |
| | pH 5.5 | |
| D5 | D4 + 0.2% m-cresol | 67.2 |
| D6 | D4 + Methyparaben 0.9 mg/mL | 69.1 |
| D7 | 20 mM L-Histidine | 68.0 |
| | 50 mM sodium chloride | |
| | Polysorbate 80 (0.05 mg/mL) | |
| | pH 5.5 | |
| D8 | D7 + 0.2% m-cresol | 66.2 |
| D9 | D7 + Methyparaben 0.9 mg/mL | 67.7 |
| D10 | 20 mM L-Histidine | 68.1 |
| | 50 mM sodium chloride | |
| | Polysorbate 20 (0.05 mg/mL) | |
| | pH 5.5 | |
| D11 | D10 + 0.2% m-cresol | 66.6 |
| D12 | D10 + Methyparaben 0.9 mg/mL | 68.1 |
| E1 | 20 mM L-Histidine | 68.2 |
| | 140 mM sodium chloride | |
| | Polysorbate 80 (0.05 mg/mL) | |
| | pH 6.5 | |
| E2 | E1 + m-cresol (0.2%) | no peak* |
| E3 | E1 + Methyparaben 0.9 mg/mL | 67.9 |
| E4 | 20 mM L-Histidine | 67.9 |
| | 140 mM sodium chloride | |
| | Polysorbate 20 (0.05 mg/mL) | |
| | pH 6.5 | |
| E5 | E4 + 0.2% m-cresol | no peak* |
| E6 | E4 + Methyparaben 0.9 mg/mL | 67.6 |
| E7 | 20 mM L-Histidine | 67.3 |
| | 50 mM sodium chloride | |
| | Polysorbate 80 (0.05 mg/mL) | |
| | pH 6.5 | |
| E8 | E7 + 0.2% m-cresol | no peak* |
| E9 | E7 + Methyparaben 0.9 mg/mL | 67.5 |
| E10 | 20 mM L-Histidine | 67.2 |
| | 50 mM sodium chloride | |
| | Polysorbate 20 (0.05 mg/mL) | |
| | pH 6.5 | |
| E11 | E10 + 0.2% m-cresol | 64.6 |
| E12 | E10 + Methyparaben 0.9 mg/mL | 66.9 |

*No peak indicates that no distinct melting point was observed.

The Tm of formulas D1, D2, D3, D4, D35, and D6 in the presence of one of each of the following sugars (1%): sucrose, trehalose, D-mannitol, maltose, and sorbitol, was further tested using DSF.

The formulations with and without sugars were also tested under stress conditions. 1 day at 40° C., followed by 1 day at 45° C., followed by 4 days at 55° C. Size exclusion HPLC analysis was used to detect and quantify monomeric and aggregation form of antibodies in the various formulations after being subjected to stress conditions. The sample was loaded onto SHODEX™ KW803 column (8 mm×300 mm) and attached to a KW-G guard column. An Agilent 1100 chromatography system was used with 2×PBS (270 mM NaCl, 5.4 mM KCl, 8.6 mM Na₂PO₄), pH 7.2 as a running buffer at a constant flow rate of 0.5 mL/min. The column was calibrated with BIORAD gel filtration standard (Catalog No. 151-1901) composed of thyroglobulin, bovine γ-globulin, chicken ovalbumin, equine myoglobin, and vitamin B12 (molecular weight 1,350-670,000). The amount of monomeric antibody remaining in solution was determined by measuring UV absorbance at 214 nm or 280 nm and calculating the peak area under the curve.

Based on DSF and HPLC analysis, formulations containing L-histidine, sodium chloride, polysorbate 80, and having a pH of between 5.0 and 6.2, such as Formulas D1, D2, D3, D4, D6, D7, D10, and D12, were considered more desirable. For example, formulations desirable for single dosing are: 20 mM L-Histidine; 140 mM sodium chloride; polysorbate 80 (0.05 mg/mL); pH 5.5. Formulations desirable for multidosing (in the presence of preservatives) are:
1. 20 mM L-Histidine; 140 mM sodium chloride; Polysorbate 80 (0.05 mg/mL); sucrose (1-3%); m-cresol (0.2%); pH 5.5; and
2. 20 mM L-Histidine; 140 mM sodium chloride; Polysorbate 80 (0.05 mg/mL); trehalose (1-3%); methylparaben (0.9%); pH 5.5.
1. 20 mM L-Histidine; 140 mM sodium chloride; Polysorbate 80 (0.05 mg/mL); sucrose (1-3%); m-cresol (0.2%); pH 5.5; and
2. 20 mM L-Histidine; 140 mM sodium chloride; Polysorbate 80 (0.05 mg/mL); trehalose (1-3%); methylparaben (0.9%); pH 5.5.

1.-60. (canceled)

61. A pharmaceutical composition comprising an anti-IL31 antibody and a pharmaceutically acceptable carrier, wherein the antibody comprises:
a) (i) a CDR-H1 comprising the amino acid sequence of SEQ ID NO: 1;
(ii) a CDR-H2 comprising the amino acid sequence of SEQ ID NO: 2, SEQ ID NO: 62, SEQ ID NO: 89, or SEQ ID NO: 87,
(iii) a CDR-H3 comprising the amino acid sequence of SEQ ID NO: 3, and
b) (i) a CDR-L1 comprising the amino acid sequence of SEQ ID NO: 8 or SEQ ID NO: 63,
(ii) a CDR-L2 comprising the amino acid sequence of SEQ ID NO: 9,
(iii) a CDR-L3 comprising the amino acid sequence of SEQ ID NO: 10, and
wherein one or more of SEQ ID NO: 1, 2, 3, 8, 9, 10, 62, 63, 87, or 89 optionally comprise an amino acid substitution.

62. The composition of claim 61, wherein the antibody comprises a heavy chain and a light chain, wherein:
a) the heavy chain comprises (i) a CDR-H1 comprising the amino acid sequence of SEQ ID NO: 1 or a variant thereof having at least 90% sequence identity to the amino acid sequence of SEQ ID NO: 1; (ii) a CDR-H2 comprising the amino acid sequence of SEQ ID NO: 2, 62, 89, or 87 or a variant thereof having at least 90% sequence identity to the amino acid sequence of SEQ ID NO: 2, SEQ ID NO: 62, SEQ ID NO: 89, or SEQ ID NO: 87; and (iii) a CDR-H3 comprising the amino acid sequence of SEQ ID NO: 3 or a variant thereof having at least 90% sequence identity to the amino acid sequence of SEQ ID NO: 3, and
b) the light chain comprises (i) a CDR-L1 comprising the amino acid sequence of SEQ ID NO: 8 or SEQ ID NO: 63 or a variant thereof having at least 90% sequence identity to the amino acid sequence of SEQ ID NO: 8 or SEQ ID NO: 63; (ii) a CDR-L2 comprising the amino acid sequence of SEQ ID NO: 9 or a variant thereof having at least 90% sequence identity to the amino acid sequence of SEQ ID NO: 9; and (iii) a CDR-L3 comprising the amino acid sequence of SEQ ID NO: 10 or a variant thereof having at least 90% sequence identity to the amino acid sequence of SEQ ID NO: 10.

63. The composition of claim 62, wherein:
a) the heavy chain comprises (i) a CDR-H1 having at least 95% sequence identity to the amino acid sequence of SEQ ID NO: 1; (ii) a CDR-H2 having at least 95% sequence identity to the amino acid sequence of SEQ ID NO: 2, SEQ ID NO: 62, SEQ ID NO: 89, or SEQ ID NO: 87; and (iii) a CDR-H3 having at least 95% sequence identity to the amino acid sequence of SEQ ID NO: 3, and
b) the light chain comprises (i) a CDR-L1 having at least 95% sequence identity to the amino acid sequence of SEQ ID NO: 8 or SEQ ID NO: 63; (ii) a CDR-L2 having at least 95% sequence identity to the amino acid sequence of SEQ ID NO: 9; and (iii) a CDR-L3 having at least 95% sequence identity to the amino acid sequence of SEQ ID NO: 10.

64. The composition of claim 61, wherein the heavy chain comprises (i) a CDR-H1 comprising the amino acid sequence of SEQ ID NO: 1; (ii) a CDR-H2 comprising the amino acid sequence of SEQ ID NO: 89; and (iii) a CDR-H3 comprising the amino acid sequence of SEQ ID NO: 3.

65. The composition of claim 61, wherein the heavy chain comprises (i) a CDR-H1 comprising the amino acid sequence of SEQ ID NO: 1; (ii) a CDR-H2 comprising the amino acid sequence of SEQ ID NO: 62 or SEQ ID NO: 87; and (iii) a CDR-H3 comprising the amino acid sequence of SEQ ID NO: 3.

66. The composition of claim 61, wherein the light chain comprises (i) a CDR-L1 comprising the amino acid sequence of SEQ ID NO: 8; (ii) a CDR-L2 comprising the amino acid sequence of SEQ ID NO: 9; and (iii) a CDR-L3 comprising the amino acid sequence of SEQ ID NO: 10.

67. The composition of claim 61, wherein the light chain comprises (i) a CDR-L1 comprising the amino acid sequence of SEQ ID NO: 63; (ii) a CDR-L2 comprising the amino acid sequence of SEQ ID NO: 9; and (iii) a CDR-L3 comprising the amino acid sequence of SEQ ID NO: 10.

68. The composition of claim 61, wherein the antibody comprises:
a) (i) a variable light chain sequence having at least 95% sequence identity to the amino acid sequence of SEQ ID NO: 24; (ii) a variable heavy chain sequence having at least 95% sequence identity to the amino acid sequence of SEQ ID NO: 25; or (iii) a variable light chain sequence as in (i) and a variable heavy chain sequence as in (ii);
b) (i) a variable light chain sequence having at least 95% sequence identity to the amino acid sequence of SEQ ID NO: 16; (ii) a variable heavy chain sequence having at least 95% sequence identity to the amino acid sequence of SEQ ID NO: 15; or (iii) a variable light chain sequence as in (i) and a variable heavy chain sequence as in (ii);
c) (i) a variable light chain sequence having at least 95% sequence identity to the amino acid sequence of SEQ ID NO: 32; (ii) a variable heavy chain sequence having at least 95% sequence identity to the amino acid sequence of SEQ ID NO: 33; or (iii) a variable light chain sequence as in (i) and a variable heavy chain sequence as in (ii);
d) (i) a variable light chain sequence having at least 95% sequence identity to the amino acid sequence of SEQ ID NO: 64; (ii) a variable heavy chain sequence having at least 95% sequence identity to the amino acid sequence of SEQ ID NO: 67; or (iii) a variable light chain sequence as in (i) and a variable heavy chain sequence as in (ii); or
e) (i) a variable light chain sequence having at least 95% sequence identity to the amino acid sequence of SEQ ID NO: 65; (ii) a variable heavy chain sequence having at least 95% sequence identity to the amino acid sequence of SEQ ID NO: 68; or (iii) a variable light chain sequence as in (i) and a variable heavy chain sequence as in (ii).

69. The composition of claim 61, wherein the antibody comprises:
a) a variable light chain sequence of SEQ ID NO: 24 and a variable heavy chain sequence of SEQ ID NO: 25;
b) a variable light chain sequence of SEQ ID NO: 16 and a variable heavy chain sequence of SEQ ID NO: 15;
c) a variable light chain sequence of SEQ ID NO: 32 and a variable heavy chain sequence of SEQ ID NO: 33;
d) a variable light chain sequence of SEQ ID NO: 64 and a variable heavy chain sequence of SEQ ID NO: 67; or
e) a variable light chain sequence of SEQ ID NO: 65 and a variable heavy chain sequence of SEQ ID NO: 68.

70. The composition of claim 61, wherein the antibody comprises a variable light chain sequence of SEQ ID NO: 32 and a variable heavy chain sequence of SEQ ID NO: 33.

71. The composition of claim 61, wherein the antibody comprises a variable light chain sequence of SEQ ID NO: 64 and a variable heavy chain sequence of SEQ ID NO: 67.

72. The composition of claim 61, wherein the antibody comprises a variable light chain sequence of SEQ ID NO: 65 and a variable heavy chain sequence of SEQ ID NO: 68.

73. The composition of claim 61, wherein the antibody comprises (a) a canine heavy chain constant region selected from an IgG-A, IgG-B, IgG-C, and IgG-D constant region; (b) a feline heavy chain constant region selected from an IgG1, IgG2a, and IgG2b constant region; or (c) an equine heavy chain constant region selected from an IgG1, IgG2, IgG3, IgG4, IgG5, IgG6 and IgG7 constant region.

74. The composition of claim 61, wherein the antibody comprises: (i) a light chain amino acid sequence of SEQ ID NO: 34; (ii) a heavy chain amino acid sequence of SEQ ID NO: 35; or (iii) a light chain amino acid sequence as in (i) and a heavy chain amino acid sequence as in (ii).

75. The composition of claim 61, wherein the antibody binds to canine IL31 with a dissociation constant (Kd) of between 5×10⁻¹¹ M and 1×10⁻¹² M, as measured by biolayer interferometry.

76. The composition of claim 61, wherein the antibody is a monoclonal antibody.

77. The composition of claim 61, wherein the antibody is a caninized, a felinized, an equinized, or a chimeric antibody.

78. The composition of claim 61, wherein the antibody is an antibody fragment selected from Fv, scFv, Fab, Fab′, F(ab′)₂, and Fab′-SH.

79. The composition of claim 61, wherein the method comprises administering in combination with the antibody:
a) a Jak inhibitor, a PI3K inhibitor, an AKT inhibitor, or a MAPK inhibitor; or
b) one or more antibodies selected from an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11α antibody, anti-IL6R antibody, anti-α4-Intergrin antibody, an anti-IL12 antibody, an anti-IL1β antibody, and an anti-BlyS antibody.

80. The composition of claim 61, wherein said amino acid substitution is a conservative substitution.

81. The composition of claim 61, wherein the pharmaceutically acceptable carrier comprises L-histidine.

82. The composition of claim 61, wherein the pharmaceutically acceptable carrier comprises sodium chloride.

83. The composition of claim 61, wherein the pharmaceutically acceptable carrier comprises L-polysorbate 80.

84. The composition of claim 61, wherein the composition has a pH of from 5.0 to 6.2.

85. The composition of claim 81, wherein the composition has an L-histidine concentration of from 5 mM to 100 mM.

86. The composition of claim 82, wherein the composition has a sodium chloride concentration of from 80 to 200 mM.

87. The composition of claim 83, wherein the composition has a L-polysorbate 80 concentration of from 0.005 mg/mL to 0.5 mg/mL.

88. The composition of claim 61, wherein the pharmaceutically acceptable carrier comprises at least one sugar.

89. The composition of claim 88, wherein the at least one sugar is present in the pharmaceutically acceptable carrier at a concentration of from 0.5% to 20%.

90. The composition of claim 61, wherein the pharmaceutically acceptable carrier comprises an ingredient selected from the group consisting of sucrose, trehalose, D-mannitol, maltose, sorbitol, and combinations thereof.

91. The composition of claim 61, wherein the pharmaceutically acceptable carrier comprises an anti-bacterial agent.

92. The composition of claim 91, wherein the anti-bacterial agent is selected from the group consisting of m-cresol, methylparaben, and combinations thereof.
